(19) United States
(12) Patent Application Publication
(10) Pub. No.: US 2022/0102005 A1
(43) Pub. Date: Mar. 31, 2022

(54) Methods and Systems for Interpreting a Diagnostic Test Result

(71) Applicant: IDEXX Laboratories, Inc., Westbrook, ME (US)

(72) Inventors: Evan Robert Grisley, Portland, ME (US); Andrea Laura Helm, Portland, ME (US); Kristen Lynn Hibbetts, Bryan, TX (US); Sara Lyn VanDeventer, Westbrook, ME (US); Richardson Charles White, JR., Portland, ME (US)

(21) Appl. No.: 17/487,782

(22) Filed: Sep. 28, 2021

Related U.S. Application Data
(60) Provisional application No. 63/084,666, filed on Sep. 29, 2020.
(60) Provisional application No. 63/185,749, filed on May 7, 2021.

(51) Int. Cl. G16H 50/20; G16H 20/10; G16H 15/00; G16H 10/60; G16H 10/40; G06F 9/451; A61B 5/00

(52) U.S. Cl. CPC G16H 50/20; G16H 20/10; G16H 15/00; G16H 10/60; A61B 2503/40; G06F 9/451; A61B 5/7475; A61B 5/4839; A61B 5/742; G16H 10/40

(57) ABSTRACT

In an example, a computer-implemented method for interpreting a diagnostic test result includes receiving a diagnostic test result for an animal patient as a result of a series of diagnostic tests performed on the animal patient, based on the diagnostic test result being indicative of a steroid analyte, initiating an automated clinical decision support interface on a graphical user interface for the diagnostic test result for the animal patient, executing a set of predetermined rules for processing the diagnostic test result for the animal patient to generate a clinical interpretation of the diagnostic test, and responsively providing via the graphical user interface the clinical interpretation of the diagnostic test. In another example, based on the diagnostic test results being indicative of an increased possibility of liver dysfunction, the set of predetermined rules are executed to generate a hepatobiliary alert for inclusion in the automated clinical decision support interface.

### CROSS REFERENCE TO RELATED APPLICATIONS

The present disclosure claims priority to U.S. provisional application number 63/084,666, filed on Sep. 29, 2020 and to U.S. provisional application number 63/185,749, filed on May 7, 2021, the entirety of each of which is herein incorporated by reference.

### FIELD

The present disclosure relates generally to methods and systems for interpreting a diagnostic test result, and more particularly, to providing programmatic clinical decision support based on a predetermined rule set for ease of understanding test results per patient.

### BACKGROUND

Many veterinarians perform diagnostic testing on animal patients as a practice to assist with routine testing and check-ups. Testing can be performed in-clinic or samples of the animal patients may be sent to an external laboratory. Typically, results of the diagnostic tests are read and interpreted manually by the veterinarians or laboratory technicians.

### SUMMARY

In many instances, interpretations of the test results can lead to questions. Sometimes, such questions lead to delay in diagnosis due to additional support required to interpret the test results. For example, veterinarians may be required to call Help Lines to speak with medical consultants for further information on the test results.

Accordingly, a more effective system is needed for providing veterinarians and laboratory technicians with automated interpretation of test results.

In an example according to the present disclosure, a computer-implemented method for interpreting a diagnostic test result is described that comprises receiving, at a computing device, a diagnostic test result for an animal patient as a result of a series of diagnostic tests performed on the animal patient, based on the diagnostic test result being indicative of a steroid analyte, the computing device programmatically initiating an automated clinical decision support interface on a graphical user interface for the diagnostic test result for the animal patient, in response to receiving a selection on the graphical user interface to initiate automated clinical decision support for the diagnostic test result for the animal patient, prompting a user via the graphical user interface to provide input regarding (i) a dose of medication provided to the animal patient for the diagnostic test, and (ii) information relating to at least one observed clinical sign in the animal patient, based on (i) the dose of medication provided to the animal patient and (ii) the information relating to the at least one observed clinical sign in the animal patient, the computing device executing a set of predetermined rules for processing the diagnostic test result for the animal patient to generate a clinical interpretation of the diagnostic test, and responsively providing via the graphical user interface the clinical interpretation of the diagnostic test.

In another example, a computing device is described comprising one or more processors, and non-transitory computer readable medium storing instructions executable by the one or more processors to perform functions. The functions comprise receiving a diagnostic test result for an animal patient as a result of a series of diagnostic tests performed on the animal patient, based on the diagnostic test result being indicative of a steroid analyte, initiating an automated clinical decision support interface on a graphical user interface for the diagnostic test result for the animal patient, in response to receiving a selection on the graphical user interface to initiate automated clinical decision support for the diagnostic test result for the animal patient, prompting a user via the graphical user interface to provide input regarding (i) a dose of medication provided to the animal patient for the diagnostic test, and (ii) information relating to at least one observed clinical sign in the animal patient, based on (i) the dose of medication provided to the animal patient and (ii) the information relating to the at least one observed clinical sign in the animal patient, executing a set of predetermined rules for processing the diagnostic test result for the animal patient to generate a clinical interpretation of the diagnostic test, and responsively providing via the graphical user interface the clinical interpretation of the diagnostic test.

In another example, a non-transitory computer readable medium is described having stored thereon instructions, that when executed by one or more processors of a computing device, cause the computing device to perform functions. The functions comprise receiving a diagnostic test result for an animal patient as a result of a series of diagnostic tests performed on the animal patient, based on the diagnostic test result being indicative of a steroid analyte, initiating an automated clinical decision support interface on a graphical user interface for the diagnostic test result for the animal patient, in response to receiving a selection on the graphical user interface to initiate automated clinical decision support for the diagnostic test result for the animal patient, prompting a user via the graphical user interface to provide input regarding (i) a dose of medication provided to the animal patient for the diagnostic test, and (ii) information relating to at least one observed clinical sign in the animal patient, based on (i) the dose of medication provided to the animal patient and (ii) the information relating to the at least one observed clinical sign in the animal patient, executing a set of predetermined rules for processing the diagnostic test result for the animal patient to generate a clinical interpretation of the diagnostic test, and responsively providing via the graphical user interface the clinical interpretation of the diagnostic test.

The features, functions, and advantages that have been discussed can be achieved independently in various examples or may be combined in yet other examples. Further details of the examples can be seen with reference to the following description and drawings.

### BRIEF DESCRIPTION OF THE FIGURES

Examples and descriptions of the present disclosure will be readily understood by reference to the following detailed description of illustrative examples when read in conjunction with the accompanying drawings, wherein:

FIG. 1 illustrates an example of a system, according to one or more embodiments shown and described herein.

FIG. 2 illustrates an example of a computing device of the system of FIG. 1, according to one or more embodiments shown and described herein.

FIG. 3 is an example illustration of a graphical user interface of the system of FIG. 1 illustrating diagnostic test results, according to one or more embodiments shown and described herein.

FIG. 4 is an example illustration of the graphical user interface of FIG. 3 illustrating diagnostic test results with a clinical decision support interface, according to one or more embodiments shown and described herein.

FIG. 5 is an example illustration of the graphical user interface of FIG. 3, illustrating diagnostic test results with the clinical decision support interface offering selections for interpretation, according to one or more embodiments shown and described herein.

FIG. 6 is another example illustration of the graphical user interface of FIG. 3 illustrating diagnostic test results with the clinical decision support interface, according to one or more embodiments shown and described herein.

FIG. 7 is another example illustration of the graphical user interface of FIG. 3 illustrating diagnostic test results with the clinical decision support interface illustrating a clinical interpretation, according to one or more embodiments shown and described herein.

FIG. 8 is another example illustration of the graphical user interface of FIG. 3 illustrating diagnostic test results with the clinical decision support interface illustrating another clinical interpretation, according to one or more embodiments shown and described herein.

FIG. 9 is another example illustration of the graphical user interface of FIG. 3 illustrating diagnostic test results with the clinical decision support interface illustrating another clinical interpretation, according to one or more embodiments shown and described herein.

FIG. 10A illustrates an example of the clinical decision support interface of FIGS. 6-9 with details for the Dexamethasone Suppression Interpretation, according to one or more embodiments shown and described herein.

FIG. 10B illustrates another example of the clinical decision support interface of FIGS. 6-9 with details for the Dexamethasone Suppression Interpretation, according to one or more embodiments shown and described herein.

FIG. 10C illustrates another example of the clinical decision support interface of FIGS. 6-9 with details for the Dexamethasone Suppression Interpretation, according to one or more embodiments shown and described herein.

FIG. 10D illustrates another example of the clinical decision support interface of FIGS. 6-9 with details for the Dexamethasone Suppression Interpretation, according to one or more embodiments shown and described herein.

FIG. 11 illustrates an example of the graphical user interface of FIG. 3 illustrating diagnostic test results with the clinical decision support interface illustrating a hepatobiliary alert, according to one or more embodiments shown and described herein.

FIG. 12 is another example illustration of the graphical user interface of FIG. 3 illustrating diagnostic test results with the clinical decision support interface illustrating details for the clinical interpretation, according to one or more embodiments shown and described herein.

FIG. 13 is another example illustration of the graphical user interface of FIG. 3 illustrating diagnostic test results with the clinical decision support interface illustrating details for the clinical interpretation, according to one or more embodiments shown and described herein.

FIG. 14 is another example illustration of the graphical user interface of FIG. 3, illustrating diagnostic test results with the clinical decision support interface illustrating the clinical interpretation, according to one or more embodiments shown and described herein.

FIG. 15A illustrates an example of the clinical decision support interface of FIGS. 6-9 with details for the hepatobiliary alert, according to one or more embodiments shown and described herein.

FIG. 15B illustrates another example of the clinical decision support interface of FIGS. 6-9 with details for the hepatobiliary alert, according to one or more embodiments shown and described herein.

FIG. 15C illustrates another example of the clinical decision support interface of FIGS. 6-9 with details for the hepatobiliary alert, according to one or more embodiments shown and described herein.

FIG. 15D illustrates another example of the clinical decision support interface of FIGS. 6-9 with details for the hepatobiliary alert, according to one or more embodiments shown and described herein.

FIG. 16A illustrates an example of the clinical decision support interface of FIGS. 6-9 with details for the urinalysis test, according to one or more embodiments shown and described herein.

FIG. 16B illustrates another example of the clinical decision support interface of FIGS. 6-9 with details for the urinalysis test, according to one or more embodiments shown and described herein.

FIG. 17 is an example illustration of the graphical user interface of FIG. 3 illustrating still other diagnostic test results with the clinical decision support interface, according to one or more embodiments shown and described herein.

FIG. 18A illustrates an example of the clinical decision support interface with details for the 4Dx alert of FIG. 17, according to one or more embodiments shown and described herein.

FIG. 18B illustrates an example of the clinical decision support interface with details for the 4Dx alert of FIG. 17, according to one or more embodiments shown and described herein.

FIG. 18C illustrates another example of the clinical decision support interface with details for the 4Dx alert of FIG. 17, according to one or more embodiments shown and described herein.

FIG. 18D illustrates another example of the clinical decision support interface with details for the 4Dx alert of FIG. 17, according to one or more embodiments shown and described herein.

FIG. 19A illustrates another example of the clinical decision support interface of FIGS. 6-9 with details for test codes related to next step considerations, according to one or more embodiments shown and described herein.

FIG. 19B illustrates an example of the graphical user interface of FIG. 3 with an ordering module, according to one or more embodiments shown and described herein.

FIG. 20 shows a flowchart of an example of a computer-implemented method for interpreting a diagnostic test result utilizing the system of FIG. 1, according to one or more embodiments shown and described herein.

### DETAILED DESCRIPTION

Examples of the present disclosure will now be described more fully hereinafter with reference to the accompanying drawings. Several different examples are described and should not be construed as limited to all possible alternatives. Rather, these examples are described so that this disclosure is thorough and complete and fully conveys a scope of the disclosure to those skilled in the art.

Within examples, computer-implemented methods for interpreting a diagnostic test result are described. A computing device receives a diagnostic test result for an animal patient as a result of a series of diagnostic tests performed on the animal patient, and then programmatically initiates an automated clinical decision support interface on a graphical user interface for the diagnostic test result for the animal patient. The clinical decision support interface offers assistance to end users for interpretation of the diagnostic test results.

The example computer-implemented methods include, in response to receiving a selection on the graphical user interface to initiate automated clinical decision support for the diagnostic test result for the animal patient, prompting a user via the graphical user interface to provide input regarding (i) a dose of medication provided to the animal patient for the diagnostic test, and (ii) information relating to at least one observed clinical sign in the animal patient, and based at least in part on (i) the dose of medication provided to the animal patient and (ii) the information relating to the at least one observed clinical sign in the animal patient, the computing device executing a set of predetermined rules for processing the diagnostic test result for the animal patient to generate a clinical interpretation of the diagnostic test. Responsively, the computing device provides, via the graphical user interface, the clinical interpretation of the diagnostic test.

The systems and methods described herein provide a solution to enable computing devices to analyze test results in a programmatic manner based on user input specific for each patient. Implementations of this disclosure provide technological improvements that are particular to computer technology, for example, those concerning analysis of diagnostic test results. Computer-specific technological problems, such as generating clinical decisions based on an analysis of diagnostic test results, can be wholly or partially solved by implementations of this disclosure. For example, implementation of embodiments described in this disclosure allows for accurate diagnosis of a patient by a computing device processing diagnostic test results in combination with additional user inputs to output a diagnosis for a specific individual patient.

The systems and methods of the present disclosure further address problems particular to computer devices, for example, those concerning post-processing of diagnostic results generally without context to a specific individual patient.

Implementations of this disclosure can thus introduce new and efficient improvements in the ways in which diagnostic test results are analyzed, resulting in workflow efficiencies due to automation of clinical decision support.

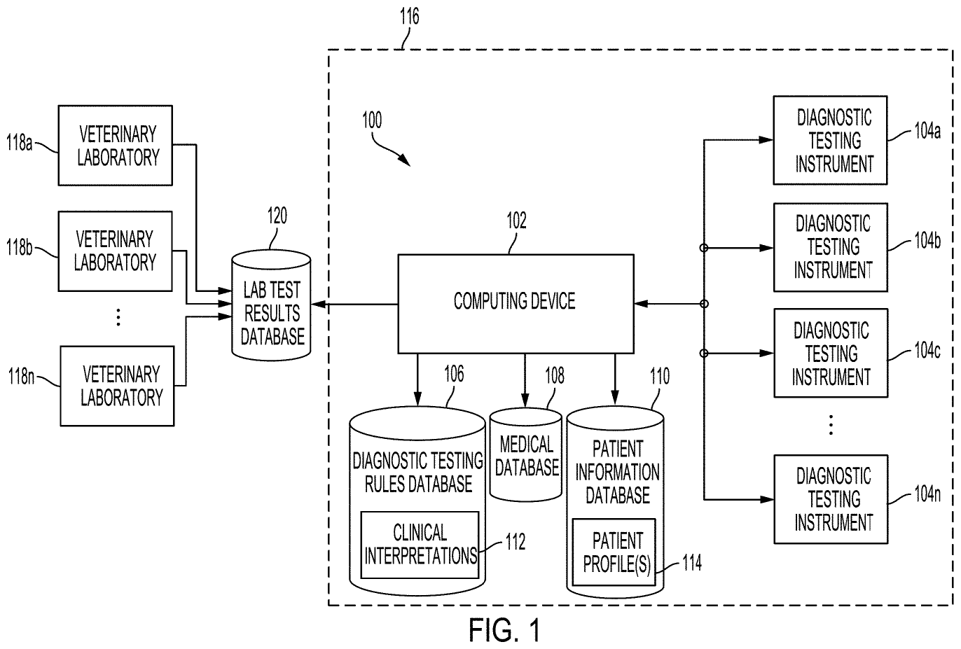

Referring now to the figures, FIG. 1 illustrates an example of a system 100, according to an example implementation. The system 100 includes a computing device 102 coupled to and in communication with one or more diagnostic testing instruments 104a-n. The computing device 102 may be in wired or wireless communication with the one or more diagnostic testing instruments 104a-n (e.g., some may be in wired Ethernet communication or may use Wi-Fi communication). In some embodiments, the system 100 may include, or components of the system 100 may be in communication with, a network (e.g., Internet) for access to cloud databases.

Although four diagnostic testing instruments are shown, more or fewer diagnostic testing instruments may be included in the system 100.

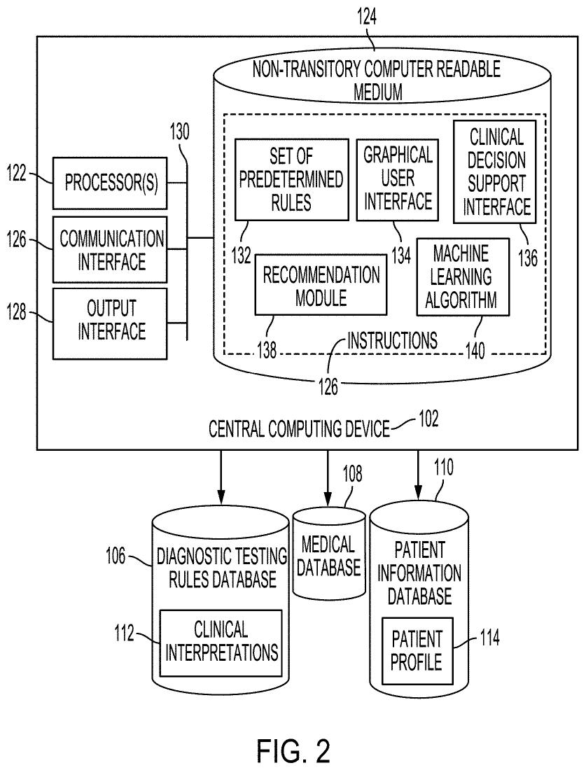

In an example, the computing device 102 is the IDEXX VetLab Station (more details of the central computing device 102 are described with reference to FIG. 2), and the diagnostic testing instruments 104a-n include veterinary analyzers operable to conduct a diagnostic test of a sample of a patient (e.g., operable to determine hemoglobin amounts in a blood sample). In one example, the computing device 102 is in communication with a veterinary analyzer of the one or more of the diagnostic testing instruments 104a-n and is operable to control operation of the veterinary analyzer. The diagnostic testing instruments 104a-n output signals, such as signals indicative of diagnostic test results or other information, to the computing device 102. Within examples, the diagnostic testing instruments 104a-n may be any one or combination of a clinical chemistry analyzer, a hematology analyzer, a urine analyzer, an immunoassay reader, a sediment analyzer, a blood analyzer, and a digital radiology machine.

In embodiments, the system 100 includes a diagnostic testing rules database 106 storing a plurality of rules for performing diagnostic testing and interpreting diagnostic test results. The diagnostic testing rules database 106 includes a set of clinical interpretations 112 of associated diagnostic tests, and each of the clinical interpretations 112 is associated with an amount of a dose of medication provided to the animal patient. Each of the clinical interpretations 112 can also be associated with observed clinical signs in the animal patient.

The system 100 further includes a medical database 108 for storing medical data including ranges of normal, low, and high test results. In embodiments, the computing device 102 is in communication with the medical database 108 for access to data within the medical database 108. In an example operation, the computing device 102 may access the medical database 108 to compare a current test result with the typical ranges for interpretation of the current test result, and the computing device 102 can send an analysis of the current test result to a display.

In some embodiments, the system 100 includes a patient information database 110 for storing patient profile(s) 114. The patient profile(s) 114 include information such as patient test records for the animal patient, and information about each patient, such as species, weight, and age, for example.

The computing device 102 is in communication with the diagnostic testing rules database 106, the medical database 108, and the patient information database 110 via a network connection (as shown in FIG. 1, which may be wired or wirelessly) or in some examples, any or all of the diagnostic testing rules database 106, the medical database 108, and the patient information database 110 may reside in the cloud and the computing device 102 can access the databases via a network.

In FIG. 1, the computing device 102 and the diagnostic testing instruments 104a-n are positioned in a location 116. The location is a veterinary laboratory, in one example, but could include any location in which the one or more diagnostic testing instruments 104a-n may be utilized.

In some examples, additional veterinary laboratories 118a-n are also present that include the same or similar diagnostic testing instruments 104a-n . A lab test results database 120 may store diagnostic test results from any or all veterinary laboratories 118a-n, as well as associated information including symptoms and follow-on testing performed in each situation. The additional veterinary laboratories 118a-n and the location 116 are each remote from each other and located at different geographic locations, in some examples, and communicate information to the lab test results database 120 over a network.

The computing device 102 may access the lab test results database 120 to learn what the other veterinary laboratories 118a-n have done in some instances and leverage success and failures of the other veterinary laboratories 118a-n when generating the recommendation for any follow-on testing.

FIG. 2 illustrates an example of the computing device 102, according to an example implementation. The computing device 102 includes one or more processor(s) 122, and non-transitory computer readable medium 124 having stored therein instructions 126 that when executed by the one or more processor(s) 122, causes the computing device 102 to perform functions for interpreting a diagnostic test result.

To perform these functions, the computing device 102 also includes a communication interface 126, an output interface 128, and each component of the computing device 102 is connected to a communication bus 130. The computing device 102 may also include hardware to enable communication within the computing device 102 and between the computing device 102 and other devices (not shown). The hardware may include transmitters, receivers, and antennas, for example. The computing device 102 may further include a display (not shown).

The communication interface 126 may be a wireless interface and/or one or more wireline interfaces that allow for both short-range communication and long-range communication to one or more networks or to one or more remote devices. Such wireless interfaces may provide for communication under one or more wireless communication protocols, Bluetooth, WiFi (e.g., an institute of electrical and electronic engineers (IEEE) 802.11 protocol), Long-Term Evolution (LTE), cellular communications, near-field communication (NFC), and/or other wireless communication protocols. Such wireline interfaces may include an Ethernet interface, a Universal Serial Bus (USB) interface, or similar interface to communicate via a wire, a twisted pair of wires, a coaxial cable, an optical link, a fiber-optic link, or other physical connection to a wireline network. Thus, the communication interface 126 may be configured to receive input data from one or more devices, and may also be configured to send output data to other devices.

The non-transitory computer readable medium 124 may include or take the form of memory, such as one or more computer-readable storage media that can be read or accessed by the one or more processor(s) 122. The non-transitory computer readable medium 124 can include volatile and/or non-volatile storage components, such as optical, magnetic, organic or other memory or disc storage, which can be integrated in whole or in part with the one or more processor(s) 122. In some examples, the non-transitory computer readable medium 124 can be implemented using a single physical device (e.g., one optical, magnetic, organic or other memory or disc storage unit), while in other examples, the non-transitory computer readable medium 124 can be implemented using two or more physical devices. The non-transitory computer readable medium 124 thus is a computer readable storage, and the instructions 126 are stored thereon. The instructions 126 include computer executable code.

The one or more processor(s) 122 may be general-purpose processors or special purpose processors (e.g., digital signal processors, application specific integrated circuits, etc.). The one or more processor(s) 122 may receive inputs from the communication interface 126 (e.g., diagnostic test results), and process the inputs to generate outputs that are stored in the non-transitory computer readable medium 124. The one or more processor(s) 122 can be configured to execute the instructions 126 (e.g., computer-readable program instructions) that are stored in the non-transitory computer readable medium 124 and are executable to provide the functionality of the central computing device 102 described herein.

The output interface 128 outputs information for transmission, reporting, or storage, and thus, the output interface 128 may be similar to the communication interface 126 and can be a wireless interface (e.g., transmitter) or a wired interface as well.

The instructions 124 may include specific software for performing the functions including a set of predetermined rules 132, a graphical user interface 134, and a clinical decision support interface 136.

The set of predetermined rules 132 are executable by the computing device 102 to generate a clinical interpretation of the diagnostic test performed on the patient. As such, the set of predetermined rules are executed based on inputs including the diagnostic test result(s) as well as other inputs including a dose of medication provided to the animal patient and information relating to at least one observed clinical sign in the animal patient.

As an example, in some embodiments the diagnostic test is a Dexamethasone Suppression Test that relates to cortisol testing. Such a test involves giving a dose of a corticosteroid medicine called dexamethasone to the animal patient to determine how it affects a level of a hormone called cortisol in the blood. The impact of the le of cortisol in the blood can be indicative of one or more conditions n the animal subject, such as Cushing's disease.

The computing device 102 receives diagnostic test results of the dexamethasone suppression test, and executes the predetermined rules 132 to generate the clinical interpretation. In some embodiments, the computing device 102 may additionally request input, such as information of the dose of medication provided to the animal patient for the diagnostic test (e.g., input regarding information indicating an amount of dexamethasone provided to the animal patient). In some embodiments, the computing device 102 requests input such as information relating to at least one observed clinical sign in the animal patient (e.g., information indicating a presence or absence of a clinical sign consistent with Cushing's disease).

Examples of the predetermined rule set are shown below in Tables 1 and 2. Table 1 illustrates a predetermined rule set for instances in which the dose of medication provided to the animal patient is “high”. Table 2 illustrates a predetermined rule set for instances in which the dose of medication provided to the animal patient is “low”. In Table 1 and Table 2, units of micrograms per deciliter (μg/dL) of whole blood are used. Both the low dose and the high dose dexamethasone suppression tests take eight (8) hours to complete and involve multiple blood samples. A first sample can be taken prior to administration of dexamethasone, and second and third samples are generally taken at four (4) and eight (8) hours following administration of dexamethasone. Differences between the low dose and high dose tests are an amount of dexamethasone that is injected. In Table 1 and Table 2, a first column indicates test results of an amount of cortisol in units of μg/dL in a blood sample after eight (8) hours, and the second columns indicates test results of the amount of cortisol in units of μg/dL in a blood sample after four (4) hours. In addition, in Table 1 and Table 2, reference to “clinical signs” refers to a behavioral or physical observation of the patient.

TABLE 1

| 8 Hr | 4 Hr | | | |
|---|---|---|---|---|
| μg/dL | μg/dL | Clinical | | |
| (US) | (US) | Signs | Text (High Dose) | Algorithm |
| ≤1.5 | Any | Yes | The result of the high dose dexamethasone | If “8 Hours |
| | | | suppression (HDDS) test in this dog supports a | Result” <=1.5 |
| | | | diagnosis of pituitary-dependent | And “Clinical |
| | | | hyperadrenocorticism. | Signs” = |
| | | | In a dog with clinical signs consistent with | “Yes”” |
| | | | hyperadrenocorticism, treatment for the disease may | |
| | | | be recommended at this time. If the dog has | |
| | | | concurrent illness (i.e. Diabetes mellitus), it should | |
| | | | be considered to first manage the concurrent disease | |
| | | | and repeat a low-dose dexamethasone suppression | |
| | | | (LDDS) test prior to beginning therapy for | |
| | | | hyperadrenocorticism. | |
| | | | Please note that this test is designed to differentiate | |
| | | | pituitary- from adrenal-dependent disease in a dog | |
| | | | that has already been diagnosed with | |
| | | | hyperadrenocorticism based on either a LDDS test | |
| | | | or an ACTH Stimulation test. | |
| >1.5 | Any | Yes | The result of the high dose dexamethasone | If (“8 Hours |
| AND | | | suppression (HDDS) test in this dog supports a | Result” >1.5 |
| <50% | | | diagnosis of pituitary-dependent | And “8 Hours |
| | | | hyperadrenocorticism. | Result” < |
| | | | In a dog with clinical signs consistent with | (“Baseline |
| | | | hyperadrenocorticism, treatment for the disease may | Result”* 0.5)) |
| | | | be recommended at this time. If the dog has | And “Clinical |
| | | | concurrent illness (i.e. Diabetes mellitus), it should | Signs” = |
| | | | be considered to first manage the concurrent disease | “Yes” |
| | | | and repeat a low-dose dexamethasone suppression | |
| | | | (LDDS) test prior to beginning therapy for | |
| | | | hyperadrenocorticism. | |
| | | | Please note that this test is designed to differentiate | |
| | | | pituitary- from adrenal-dependent disease in a dog | |
| | | | that has already been diagnosed with | |
| | | | hyperadrenocorticism based on either a LDDS test | |
| | | | or an ACTH Stimulation test. | |
| >1.5 | ≤1.5 | Yes | The result of the high dose dexamethasone | If (“8 Hours |
| AND | | | suppression (HDDS) test in this dog supports a | Result” >1.5 |
| ≥50% | | | diagnosis of pituitary-dependent | And “8 Hours |
| | | | hyperadrenocorticism. | Result” >= |
| | | | In a dog with clinical signs consistent with | (“Baseline |
| | | | hyperadrenocorticism, treatment for the disease may | Result”* 0.5)) |
| | | | be recommended at this time. If the dog has | And “4 Hours |
| | | | concurrent illness (i.e. Diabetes mellitus), it should | Result” <=1.5 |
| | | | be considered to first manage the concurrent disease | And “Clinical |
| | | | and repeat a low-dose dexamethasone suppression | Signs” = |
| | | | (LDDS) test prior to beginning therapy for | “Yes” |
| | | | hyperadrenocorticism. | |
| | | | Please note that this test is designed to differentiate | |
| | | | pituitary- from adrenal-dependent disease in a dog | |
| | | | that has already been diagnosed with | |
| | | | hyperadrenocorticism based on either a LDDS test | |
| | | | or an ACTH Stimulation test. | |
| >1.5 | >1.5 | YES | The result of the high dose dexamethasone | (“8 Hours |
| AND | AND | | suppression (HDDS) test in this dog supports a | Result” >1.5 |
| ≥50% | ≤50% | | diagnosis of pituitary-dependent | And “8 Hours |
| | | | hyperadrenocorticism. | Result” >= |
| | | | In a dog with clinical signs consistent with | (“Baseline |
| | | | hyperadrenocorticism, treatment for the disease may | Result”* 0.5)) |
| | | | be recommended at this time. If the dog has | And (“4 Hours |
| | | | concurrent illness (i.e. Diabetes mellitus), it should | Result” >1.5 |
| | | | be considered to first manage the concurrent disease | And “4 Hours |
| | | | and repeat a low-dose dexamethasone suppression | Result” <= |
| | | | (LDDS) test prior to beginning therapy for | (“Baseline |
| | | | hyperadrenocorticism. | Result”* 0.5)) |
| | | | Please note that this test is designed to differentiate | And “Clinical |
| | | | pituitary- from adrenal-dependent disease in a dog | Signs” = |
| | | | that has already been diagnosed with | “Yes” |
| | | | hyperadrenocorticism based on either a LDDS test | |
| | | | or an ACTH Stimulation test. | |
| ≤1.5 | Any | NO | The result of the high dose dexamethasone | If “8 Hours |
| | | | suppression (HDDS) test in this dog may support a | Result” <=1.5 |
| | | | diagnosis of pituitary-dependent | And “Clinical |
| | | | hyperadrenocorticism, however in a dog without | Signs” = |
| | | | clinical signs consistent with | “No” |
| | | | hyperadrenocorticism, non-adrenal illness or stress | |
| | | | at the time of the test may influence the results. If the | |
| | | | dog has concurrent illness, it is recommended to first | |
| | | | manage the concurrent disease prior to further | |
| | | | assessment for hyperadrenocorticism. | |
| | | | Hyperadrenocorticism is considered a clinical | |
| | | | syndrome. If the patient does not have clinical signs | |
| | | | consistent with hyperadrenocorticism, treatment is | |
| | | | not typically recommended. | |
| >1.5 | Any | NO | The result of the high dose dexamethasone | If (“8 Hours |
| AND | | | suppression (HDDS) test in this dog may support a | Result” >1.5 |
| <50% | | | diagnosis of pituitary-dependent | And “8 Hours |
| | | | hyperadrenocorticism, however in a dog without | Result” < |
| | | | clinical signs consistent with | (“Baseline |
| | | | hyperadrenocorticism, non-adrenal illness or stress | Result”* 0.5)) |
| | | | at the time of the test may influence the results. If the | And “Clinical |
| | | | dog has concurrent illness, it is recommended to first | Signs” = |
| | | | manage the concurrent disease prior to further | “No” |
| | | | assessment for hyperadrenocorticism. | |
| | | | Hyperadrenocorticism is considered a clinical | |
| | | | syndrome. If the patient does not have clinical signs | |
| | | | consistent with hyperadrenocorticism, treatment is | |
| | | | not typically recommended. | |
| >1.5 | ≤1.5 | NO | The result of the high dose dexamethasone | If (“8 Hours |
| AND | | | suppression (HDDS) test in this dog may support a | Result” >1.5 |
| ≥50% | | | diagnosis of pituitary-dependent | And “8 Hours |
| | | | hyperadrenocorticism, however in a dog without | Result” >= |
| | | | clinical signs consistent with | (“Baseline |
| | | | hyperadrenocorticism, non-adrenal illness or stress | Result”* 0.5)) |
| | | | at the time of the test may influence the results. If the | And “4 Hours |
| | | | dog has concurrent illness, it is recommended to first | Result” <=1.5 |
| | | | manage the concurrent disease prior to further | And “Clinical |
| | | | assessment for hyperadrenocorticism. | Signs” = |
| | | | Hyperadrenocorticism is considered a clinical | “No” |
| | | | syndrome. If the patient does not have clinical signs | |
| | | | consistent with hyperadrenocorticism, treatment is | |
| | | | not typically recommended. | |
| >1.5 | >1.5 | NO | The result of the high dose dexamethasone | If (“8 Hours |
| AND | AND | | suppression (HDDS) test in this dog may support a | Result” >1.5 |
| ≥50% | ≤50% | | diagnosis of pituitary-dependent | And “8 Hours |
| | | | hyperadrenocorticism, however in a dog without | Result” >= |
| | | | clinical signs consistent with | (“Baseline |
| | | | hyperadrenocorticism, non-adrenal illness or stress | Result”* 0.5)) |
| | | | at the time of the test may influence the results. If the | And (“4 Hours |
| | | | dog has concurrent illness, it is recommended to first | Result” >1.5 |
| | | | manage the concurrent disease prior to further | And “4 Hours |
| | | | assessment for hyperadrenocorticism. | Result” <= |
| | | | Hyperadrenocorticism is considered a clinical | (“Baseline |
| | | | syndrome. If the patient does not have clinical signs | Result”* 0.5)) |
| | | | consistent with hyperadrenocorticism, treatment is | And “Clinical |
| | | | not typically recommended. | Signs” = |
| | | | | “No” |
| >1.5 | >1.5 | YES | The result of the high dose dexamethasone | If (“8 Hours |
| AND | AND | | suppression (HDDS) test in this dog does | Result” >1.5 |
| ≥50% | >50% | | not differentiate pituitary-dependent from adrenal- | And “8 Hours |
| | | | dependent hyperadrenocorticism. | Result” >= |
| | | | In a dog with clinical signs consistent with | (“Baseline |
| | | | hyperadrenocorticism, it is recommended to attempt | Result”* 0.5)) |
| | | | differentiation of pituitary-dependent from adrenal- | And (“4 Hours |
| | | | dependent disease by performing either an | Result” >1.5 |
| | | | abdominal ultrasound, computed tomography (CT), | And “4 Hours |
| | | | magnetic resonance imaging (MRI), and/or an | Result” > |
| | | | endogenous ACTH concentration. If the dog has | (“Baseline |
| | | | concurrent illness (i.e. Diabetes mellitus), it should | Result”* 0.5)) |
| | | | be considered to first manage the concurrent disease | And “Clinical |
| | | | and repeat a low-dose dexamethasone suppression | Signs” = |
| | | | (LDDS) test prior to performing additional | “Yes” |
| | | | differentiating tests. | |
| | | | Please note that this test is designed to differentiate | |
| | | | pituitary- from adrenal-dependent disease in a dog | |
| | | | that has already been diagnosed with | |
| | | | hyperadrenocorticism based on either a LDDS test | |
| | | | or an ACTH Stimulation test. | |
| >1.5 | >1.5 | NO | The result of the high dose dexamethasone | If (“8 Hours |
| AND | AND | | suppression (HDDS) test in this dog does | Result” >1.5 |
| ≥50% | >50% | | not differentiate pituitary-dependent from adrenal- | And “8 Hours |
| | | | dependent hyperadrenocorticism. | Result” >= |
| | | | In a dog without clinical signs consistent with | (“Baseline |
| | | | hyperadrenocorticism, attempted differentiation of | Result”* 0.5)) |
| | | | pituitary-dependent from adrenal-dependent and | And (“4 Hours |
| | | | treatment would typically not be considered at this | Result” >1.5 |
| | | | time. | And “4 Hours |
| | | | Hyperadrenocorticism is considered a clinical | Result”> |
| | | | syndrome. If the patient does not have clinical signs | (“Baseline |
| | | | consistent with hyperadrenocorticism, treatment is | Result”* 0.5)) |
| | | | not typically recommended. If further | And “Clinical |
| | | | differentiation is preferred, it is recommended to | Signs” = |
| | | | perform either an abdominal ultrasound, computed | “No” |
| | | | tomography (CT), magnetic resonance imaging | |
| | | | (MRI), and/or an endogenous ACTH. | |
| — | — | YES/NO | For anything else - | |
| | | | Inconclusive. Please check values. | |

TABLE 2

| 8 Hr | 4 Hr | | | |
|---|---|---|---|---|
| μg/dL | μg/dL | Clinical | | |
| (US) | (US) | Signs | Text (Low Dose) | Algorithm |
| <1 | <1 | YES | The result of the low dose dexamethasone | If “8 Hours |
| | | | suppression (LDDS) test in this dog does not | Result” <1 |
| | | | support a diagnosis of hyperadrenocorticism. | And “4 Hours |
| | | | In a dog with clinical signs consistent with | Result” <1 |
| | | | hyperadrenocorticism, it is recommended to rule | And “Clinical |
| | | | out non-adrenal causes for these clinical signs. If | Signs” = |
| | | | an alternate cause for the clinical signs is not | “Yes” |
| | | | identified, consider performing an ACTH | |
| | | | Stimulation test or repeating the LDDS test in 1- | |
| | | | 3 months. | |
| <1 | 1-1.5 | YES | The result of the low dose dexamethasone | If (“8 Hours |
| | AND | | suppression (LDDS) test in this dog does not | Result” <1 |
| | <50% | | support a diagnosis of hyperadrenocorticism. | And (“4 Hours |
| | | | In a dog with clinical signs consistent with | Result” >=1 |
| | | | hyperadrenocorticism, it is recommended to rule | And “4 Hours |
| | | | out non-adrenal causes for these clinical signs. If | Result” <=1.5)) |
| | | | an alternate cause for the clinical signs is not | And (“4 Hours |
| | | | identified, consider performing an ACTH | Result” < |
| | | | Stimulation test or repeating the LDDS test in 1- | (“Baseline |
| | | | 3 months. | Result”* 0.5)) |
| | | | | And “Clinical |
| | | | | Signs” = |
| | | | | “Yes” |
| 1-1.5 | <1 | YES | The result of the low dose dexamethasone | If (“8 Hours |
| AND | | | suppression (LDDS) test in this dog does not | Result” >=1 |
| <50% | | | support a diagnosis of hyperadrenocorticism. | And “8 Hours |
| | | | In a dog with clinical signs consistent with | Result” <=1.5) |
| | | | hyperadrenocorticism, it is recommended to rule | And (“8 Hours |
| | | | out non-adrenal causes for these clinical signs. If | Result” < |
| | | | an alternate cause for the clinical signs is not | (“Baseline |
| | | | identified, consider performing an ACTH | Result”*0.5)) |
| | | | Stimulation test or repeating the LDDS test in 1- | And (“4 Hours |
| | | | 3 months. | Result” <1) |
| | | | | And “Clinical |
| | | | | Signs” = |
| | | | | “Yes” |
| 1-1.5 | 1-1.5 | YES | The result of the low dose dexamethasone | If (“8 Hours |
| AND | AND | | suppression (LDDS) test in this dog does not | Result” >=1 |
| <50% | <50% | | support a diagnosis of hyperadrenocorticism. | And “8 Hours |
| | | | In a dog with clinical signs consistent with | Result” <=1.5) |
| | | | hyperadrenocorticism, it is recommended to rule | And (“8 Hours |
| | | | out non-adrenal causes for these clinical signs. If | Result” < |
| | | | an alternate cause for the clinical signs is not | (“Baseline |
| | | | identified, consider performing an ACTH | Result”* 0.5)) |
| | | | Stimulation test or repeating the LDDS test in 1- | And (“4 Hours |
| | | | 3 months. | Result” >=1 |
| | | | | And “4 Hours |
| | | | | Result” <=1.5) |
| | | | | And (“4 Hours |
| | | | | Result” < |
| | | | | (“Baseline |
| | | | | Result”* 0.5)) |
| | | | | And “Clinical |
| | | | | Signs” = |
| | | | | “Yes” |
| <1 | <1 | NO | The result of the low dose dexamethasone | If “8 Hours |
| | | | suppression (LDDS) test in this dog does not | Result” <1 |
| | | | support a diagnosis of hyperadrenocorticism. | And “4 Hours |
| | | | In a dog without clinical signs consistent with | Result” <1 |
| | | | hyperadrenocorticism, the disease is unlikely. In | And “Clinical |
| | | | a dog with abnormal laboratory results, it is | Signs” = |
| | | | recommended to rule out alternate causes for | “No” |
| | | | these abnormalities. No further testing for | |
| | | | hyperadrenocorticism is necessary at this time. | |
| <1 | 1-1.5 | NO | The result of the low dose dexamethasone | If (“8 Hours |
| | AND | | suppression (LDDS) test in this dog does not | Result” <1 |
| | <50% | | support a diagnosis of hyperadrenocorticism. | And (“4 Hours |
| | | | In a dog without clinical signs consistent with | Result” >=1 |
| | | | hyperadrenocorticism, the disease is unlikely. In | And “4 Hours |
| | | | a dog with abnormal laboratory results, it is | Result” <=1.5)) |
| | | | recommended to rule out alternate causes for | And (“4 Hours |
| | | | these abnormalities. No further testing for | Result” < |
| | | | hyperadrenocorticism is necessary at this time. | (“Baseline |
| | | | | Result”* 0.5)) |
| | | | | And “Clinical |
| | | | | Signs” = |
| | | | | “No” |
| 1-1.5 | <1 | NO | The result of the low dose dexamethasone | If (“8 Hours |
| AND | | | suppression (LDDS) test in this dog does not | Result” >=1 |
| <50% | | | support a diagnosis of hyperadrenocorticism. | And “8 Hours |
| | | | In a dog without clinical signs consistent with | Result” <=1.5) |
| | | | hyperadrenocorticism, the disease is unlikely. In | And (“8 Hours |
| | | | a dog with abnormal laboratory results, it is | Result” < |
| | | | recommended to rule out alternate causes for | (“Baseline |
| | | | these abnormalities. No further testing for | Result”* 0.5)) |
| | | | hyperadrenocorticism is necessary at this time. | And (“4 Hours |
| | | | | Result” <1) |
| | | | | And “Clinical |
| | | | | Signs” = |
| | | | | “No” |
| 1-1.5 | 1-1.5 | NO | The result of the low dose dexamethasone | If (“8 Hours |
| AND | AND | | suppression (LDDS) test in this dog does not | Result” >=1 |
| <50% | <50% | | support a diagnosis of hyperadrenocorticism. | And “8 Hours |
| | | | In a dog without clinical signs consistent with | Result” <=1.5) |
| | | | hyperadrenocorticism, the disease is unlikely. In | And (“8 Hours |
| | | | a dog with abnormal laboratory results, it is | Result” < |
| | | | recommended to rule out alternate causes for | (“Baseline |
| | | | these abnormalities. No further testing for | Result”* 0.5)) |
| | | | hyperadrenocorticism is necessary at this time. | And (“4 Hours |
| | | | | Result” >=1 |
| | | | | And “4 Hours |
| | | | | Result” <=1.5) |
| | | | | And (“4 Hours |
| | | | | Result” < |
| | | | | (“Baseline |
| | | | | Result”* 0.5)) |
| | | | | And “Clinical |
| | | | | Signs” = |
| | | | | “No” |
| <1 | 1-1.5 | YES | The result of the low dose dexamethasone | If (“8 Hours |
| | AND | | suppression (LDDS) test in this dog | Result” <1 |
| | ≥50% | | is inconclusive and does not rule out a diagnosis | And (“4 Hours |
| | | | of hyperadrenocorticism. | Result” >=1 |
| | | | In a dog with clinical signs consistent with | And “4 Hours |
| | | | hyperadrenocorticism, it is recommended to rule | Result” <=1.5)) |
| | | | out non-adrenal causes for these clinical signs. If | And (“4 Hours |
| | | | an alternate cause for the clinical signs is not | Result” >= |
| | | | identified, additional testing for | (“Baseline |
| | | | hyperadrenocorticism should be considered, | Result”* 0.5)) |
| | | | including an ACTH Stimulation test and | And “Clinical |
| | | | abdominal ultrasound. | Signs” = |
| | | | | “Yes” |
| <1 | >1.5 | YES | The result of the low dose dexamethasone | If (“8 Hours |
| | | | suppression (LDDS) test in this dog | Result” <1) |
| | | | is inconclusive and does not rule out a diagnosis | And (“4 Hours |
| | | | of hyperadrenocorticism. | Result” >1.5) |
| | | | In a dog with clinical signs consistent with | And “Clinical |
| | | | hyperadrenocorticism, it is recommended to rule | Signs” = |
| | | | out non-adrenal causes for these clinical signs. If | “Yes” |
| | | | an alternate cause for the clinical signs is not | |
| | | | identified, additional testing for | |
| | | | hyperadrenocorticism should be considered, | |
| | | | including an ACTH Stimulation test and | |
| | | | abdominal ultrasound. | |
| 1-1.5 | 1-1.5 | YES | The result of the low dose dexamethasone | If (“8 Hours |
| AND | AND | | suppression (LDDS) test in this dog | Result” >=1 |
| <50% | ≥50% | | is inconclusive and does not rule out a diagnosis | And “8 Hours |
| | | | of hyperadrenocorticism. | Result” <=1.5) |
| | | | In a dog with clinical signs consistent with | And (“8 Hours |
| | | | hyperadrenocorticism, it is recommended to rule | Result” < |
| | | | out non-adrenal causes for these clinical signs. If | (“Baseline |
| | | | an alternate cause for the clinical signs is not | Result”* 0.5)) |
| | | | identified, additional testing for | And (“4 Hours |
| | | | hyperadrenocorticism should be considered, | Result” >=1 |
| | | | including an ACTH Stimulation test and | And “4 Hours |
| | | | abdominal ultrasound. | Result” <=1.5) |
| | | | | And (“4 Hours |
| | | | | Result”>= |
| | | | | (“Baseline |
| | | | | Result”* 0.5)) |
| | | | | And “Clinical |
| | | | | Signs” = |
| | | | | “Yes” |
| 1-1.5 | >1.5 | YES | The result of the low dose dexamethasone | If (“8 Hours |
| AND | | | suppression (LDDS) test in this dog | Result” >=1 |
| <50% | | | is inconclusive and does not rule out a diagnosis | And “8 Hours |
| | | | of hyperadrenocorticism. | Result” <=1.5) |
| | | | In a dog with clinical signs consistent with | And (“8 Hours |
| | | | hyperadrenocorticism, it is recommended to rule | Result” < |
| | | | out non-adrenal causes for these clinical signs. If | (“Baseline |
| | | | an alternate cause for the clinical signs is not | Result”* 0.5)) |
| | | | identified, additional testing for | And (“4 Hours |
| | | | hyperadrenocorticism should be considered, | Result” >=1.5) |
| | | | including an ACTH Stimulation test and | And “Clinical |
| | | | abdominal ultrasound. | Signs” = |
| | | | | “Yes” |
| 1-1.5 | <1 | YES | The result of the low dose dexamethasone | If (“8 Hours |
| AND | | | suppression (LDDS) test in this dog | Result” >=1 |
| ≥50% | | | is inconclusive and does not rule out a diagnosis | And “8 Hours |
| | | | of hyperadrenocorticism. | Result” <=1.5) |
| | | | In a dog with clinical signs consistent with | And “8 Hours |
| | | | hyperadrenocorticism, it is recommended to rule | Result” >= |
| | | | out non-adrenal causes for these clinical signs. If | (“Baseline |
| | | | an alternate cause for the clinical signs is not | Result”* 0.5) |
| | | | identified, additional testing for | And (“4 hours |
| | | | hyperadrenocorticism should be considered, | result” <1) |
| | | | including an ACTH Stimulation test and | And “Clinical |
| | | | abdominal ultrasound. | Signs” = |
| | | | | “Yes” |
| <1 | 1-1.5 | NO | The result of the low dose dexamethasone | If (“8 Hours |
| | AND | | suppression (LDDS) test in this dog | Result” <1 |
| | ≥50% | | is inconclusive and does not rule out a diagnosis | And (“4 Hours |
| | | | of hyperadrenocorticism. | Result” >=1 |
| | | | In a dog without clinical signs consistent with | And “4 Hours |
| | | | hyperadrenocorticism, no further testing for this | Result” <=1.5)) |
| | | | disease is necessary at this time. In a dog with | And (“4 Hours |
| | | | abnormal laboratory results, it is recommended | Result” >= |
| | | | to rule out alternate causes for these | (“Baseline |
| | | | abnormalities. | Result”* 0.5)) |
| | | | | And “Clinical |
| | | | | Signs” = |
| | | | | “No” |
| <1 | >1.5 | NO | The result of the low dose dexamethasone | if (“8 Hours |
| | | | suppression (LDDS) test in this dog | Result” <1) |
| | | | is inconclusive and does not rule out a diagnosis | And (“4 Hours |
| | | | of hyperadrenocorticism. | Result” >1.5) |
| | | | In a dog without clinical signs consistent with | And “Clinical |
| | | | hyperadrenocorticism, no further testing for this | Signs” = |
| | | | disease is necessary at this time. In a dog with | “No” |
| | | | abnormal laboratory results, it is recommended | |
| | | | to rule out alternate causes for these | |
| | | | abnormalities. | |
| 1-1.5 | 1-1.5 | NO | The result of the low dose dexamethasone | If (“8 Hours |
| AND | AND | | suppression (LDDS) test in this dog | Result” >=1 |
| <50% | ≥50% | | is inconclusive and does not rule out a diagnosis | And “8 Hours |
| | | | of hyperadrenocorticism. | Result” <=1.5) |
| | | | In a dog without clinical signs consistent with | And (“8 Hours |
| | | | hyperadrenocorticism, no further testing for this | Result” < |
| | | | disease is necessary at this time. In a dog with | (“Baseline |
| | | | abnormal laboratory results, it is recommended | Result”* 0.5)) |
| | | | to rule out alternate causes for these | And (“4 Hours |
| | | | abnormalities. | Result” >=1 |
| | | | | And “4 Hours |
| | | | | Result” <=1.5) |
| | | | | And (“4 Hours |
| | | | | Result” >= |
| | | | | (“Baseline |
| | | | | Result”* 0.5)) |
| | | | | And “Clinical |
| | | | | Signs” = |
| | | | | “No” |
| 1-1.5 | >1.5 | NO | The result of the low dose dexamethasone | If (“8 Hours |
| AND | | | suppression (LDDS) test in this dog | Result” >=1 |
| <50% | | | is inconclusive and does not rule out a diagnosis | And “8 Hours |
| | | | of hyperadrenocorticism. | Result” <=1.5) |
| | | | In a dog without clinical signs consistent with | And (“8 Hours |
| | | | hyperadrenocorticism, no further testing for this | Result” < |
| | | | disease is necessary at this time. In a dog with | (“Baseline |
| | | | abnormal laboratory results, it is recommended | Result”* 0.5)) |
| | | | to rule out alternate causes for these | And (“4 Hours |
| | | | abnormalities. | Result” >=1.5) |
| | | | | And “Clinical |
| | | | | Signs” = |
| | | | | “No” |
| 1-1.5 | <1 | NO | The result of the low dose dexamethasone | If (“8 Hours |
| AND | | | suppression (LDDS) test in this dog | Result” >=1 |
| ≥50% | | | is inconclusive and does not rule out a diagnosis | And “8 Hours |
| | | | of hyperadrenocorticism. | Result” <=1.5) |
| | | | In a dog without clinical signs consistent with | And “8 Hours |
| | | | hyperadrenocorticism, no further testing for this | Result” >= |
| | | | disease is necessary at this time. In a dog with | (“Baseline |
| | | | abnormal laboratory results, it is recommended | Result”* 0.5) |
| | | | to rule out alternate causes for these | And (“4 hours |
| | | | abnormalities. | result” <1) |
| | | | | And “Clinical |
| | | | | Signs” = |
| | | | | “No” |
| >1.5 | Any | YES | The result of the low dose dexamethasone | If (“8 Hours |
| AND | Number | | suppression (LDDS) test in this dog supports a | Result” >1.5 |
| <50% | | | diagnosis of pituitary-dependent | And “8 Hours |
| | | | hyperadrenocorticism. | Result” < |
| | | | In a dog with clinical signs consistent with | (“Baseline |
| | | | hyperadrenocorticism, treatment for the disease | Result”* 0.5)) |
| | | | may be considered at this time. If the dog has | And “Clinical |
| | | | concurrent illness (i.e. diabetes mellitus), | Signs” = |
| | | | consider first managing the concurrent disease | “Yes” |
| | | | and then repeating the LDDS prior to beginning | |
| | | | therapy for hyperadrenocorticism. | |
| | | | Please note that administration of exogenous | |
| | | | steroids or stress related to concurrent illness | |
| | | | may affect the results and interpretation of the | |
| | | | dexamethasone suppression test. | |
| >1.5 | <1 | YES | The result of the low dose dexamethasone | If (“8 Hours |
| AND | | | suppression (LDDS) test in this dog supports a | Result” >1.5 |
| ≥50% | | | diagnosis of pituitary-dependent | And “8 Hours |
| | | | hyperadrenocorticism. | Result” >= |
| | | | In a dog with clinical signs consistent with | (“Baseline |
| | | | hyperadrenocorticism, treatment for the disease | Result”* 0.5)) |
| | | | may be considered at this time. If the dog has | And “4 Hours |
| | | | concurrent illness (i.e. diabetes mellitus), | Result” <1 |
| | | | consider first managing the concurrent disease | And “Clinical |
| | | | and then repeating the LDDS prior to beginning | Signs” = |
| | | | therapy for hyperadrenocorticism. | “Yes” |
| | | | Please note that administration of exogenous | |
| | | | steroids or stress related to concurrent illness | |
| | | | may affect the results and interpretation of the | |
| | | | dexamethasone suppression test. | |
| >1.5 | ≥1 | YES | The result of the low dose dexamethasone | If (“8 Hours |
| AND | AND | | suppression (LDDS) test in this dog supports a | Result” >1.5 |
| ≥50% | <50% | | diagnosis of pituitary-dependent | And “8 Hours |
| | | | hyperadrenocorticism. | Result” >= |
| | | | In a dog with clinical signs consistent with | (“Baseline |
| | | | hyperadrenocorticism, treatment for the disease | Result”* 0.5)) |
| | | | may be considered at this time. If the dog has | And (“4 Hours |
| | | | concurrent illness (i.e. diabetes mellitus), | Result” >=1 |
| | | | consider first managing the concurrent disease | And “4 Hours |
| | | | and then repeating the LDDS prior to beginning | Result” < |
| | | | therapy for hyperadrenocorticism. | (“Baseline |
| | | | Please note that administration of exogenous | Result”* 0.5)) |
| | | | steroids or stress related to concurrent illness | And “Clinical |
| | | | may affect the results and interpretation of the | Signs” = |
| | | | dexamethasone suppression test. | “Yes” |
| >1.5 | Any | NO | The result of the low dose dexamethasone | If (“8 Hours |
| AND | Number | | suppression (LDDS) test in this dog may | Result” >1.5 |
| <50% | | | support a diagnosis of pituitary-dependent | And “8 Hours |
| | | | hyperadrenocorticism, however in a dog without | Result” < |
| | | | clinical signs consistent with | (“Baseline |
| | | | hyperadrenocorticism, non-adrenal illness or | Result”* 0.5)) |
| | | | stress at the time of the test may influence the | And “Clinical |
| | | | results. If the dog has concurrent illness, it is | Signs” = |
| | | | recommended to first manage the concurrent | “No” |
| | | | disease prior to further assessment for | |
| | | | hyperadrenocorticism. | |
| | | | Hyperadrenocorticism is considered a clinical | |
| | | | syndrome. If the patient does not have clinical | |
| | | | signs consistent with hyperadrenocorticism, | |
| | | | treatment is not typically recommended. | |
| | | | It is important to consider that administration of | |
| | | | exogenous steroids or stress related to concurrent | |
| | | | illness may affect the results and interpretation of | |
| | | | the dexamethasone suppression test. | |
| >1.5 | <1 | NO | The result of the low dose dexamethasone | If (“8 Hours |
| AND | | | suppression (LDDS) test in this dog may | Result” >1.5 |
| ≥50% | | | support a diagnosis of pituitary-dependent | And “8 Hours |
| | | | hyperadrenocorticism, however in a dog without | Result” >= |
| | | | clinical signs consistent with | (“Baseline |
| | | | hyperadrenocorticism, non-adrenal illness or | Result”* 0.5)) |
| | | | stress at the time of the test may influence the | And “4 Hours |
| | | | results. If the dog has concurrent illness, it is | Result” <1 |
| | | | recommended to first manage the concurrent | And “Clinical |
| | | | disease prior to further assessment for | Signs” = |
| | | | hyperadrenocorticism. | “No” |
| | | | Hyperadrenocorticism is considered a clinical | |
| | | | syndrome. If the patient does not have clinical | |
| | | | signs consistent with hyperadrenocorticism, | |
| | | | treatment is not typically recommended. | |
| | | | It is important to consider that administration of | |
| | | | exogenous steroids or stress related to concurrent | |
| | | | illness may affect the results and interpretation of | |
| | | | the dexamethasone suppression test. | |
| >1.5 | ≥1 | NO | The result of the low dose dexamethasone | If (“8 Hours |
| AND | AND | | suppression (LDDS) test in this dog may | Result” >1.5 |
| ≥50% | <50% | | support a diagnosis of pituitary-dependent | And “8 Hours |
| | | | hyperadrenocorticism, however in a dog without | Result” >= |
| | | | clinical signs consistent with | (“Baseline |
| | | | hyperadrenocorticism, non-adrenal illness or | Result”* 0.5)) |
| | | | stress at the time of the test may influence the | And (“4 Hours |
| | | | results. If the dog has concurrent illness, it is | Result” >=1 |
| | | | recommended to first manage the concurrent | And “4 Hours |
| | | | disease prior to further assessment for | Result” < |
| | | | hyperadrenocorticism. | (“Baseline |
| | | | Hyperadrenocorticism is considered a clinical | Result”* 0.5)) |
| | | | syndrome. If the patient does not have clinical | And “Clinical |
| | | | signs consistent with hyperadrenocorticism, | Signs” = |
| | | | treatment is not typically recommended. | “No” |
| | | | It is important to consider that administration of | |
| | | | exogenous steroids or stress related to concurrent | |
| | | | illness may affect the results and interpretation of | |
| | | | the dexamethasone suppression test. | |
| >1.5 | ≥1 | YES | The result of the low dose dexamethasone | If (“8 Hours |
| AND | AND | | suppression (LDDS) test in this dog supports a | Result” >1.5 |
| ≥50% | ≥50% | | diagnosis of hyperadrenocorticism and does | And “8 Hours |
| | | | not differentiate pituitary-dependent from | Result” >= |
| | | | adrenal-dependent disease. | (“Baseline |
| | | | In a dog with clinical signs consistent with | Result”* 0.5)) |
| | | | hyperadrenocorticism, it is recommended to | And (“4 Hours |
| | | | pursue differentiation of pituitary-dependent | Result” >=1 |
| | | | from adrenal-dependent disease by performing | And “4 Hours |
| | | | either an abdominal ultrasound, high-dose | Result” >= |
| | | | dexamethasone suppression (HDDS) test, and/or | (“Baseline |
| | | | an endogenous ACTH concentration. If the dog | Result”* 0.5)) |
| | | | has concurrent illness (i.e. diabetes mellitus), | And “Clinical |
| | | | consider first managing the concurrent disease | Signs” = |
| | | | and then repeating the LDDS prior to performing | “Yes” |
| | | | additional differentiating tests. | |
| | | | Please note that administration of exogenous | |
| | | | steroids or stress related to concurrent illness | |
| | | | may affect the results and interpretation of the | |
| | | | dexamethasone suppression test. | |
| >1.5 | ≥1 | NO | The result of the low dose dexamethasone | If (“8 Hours |
| AND | AND | | suppression (LDDS) test in this dog may | Result” >1.5 |
| ≥50% | ≥50% | | support a diagnosis of hyperadrenocorticism, | And “8 Hours |
| | | | however in a dog without clinical signs | Result” >= |
| | | | consistent with hyperadrenocorticism, non- | (“Baseline |
| | | | adrenal illness or stress at the time of the test | Result”* 0.5)) |
| | | | may influence the results. If the dog has | And (“4 Hours |
| | | | concurrent illness, it is recommended to first | Result” >=1 |
| | | | manage the concurrent disease prior to further | And “4 Hours |
| | | | assessment for and differentiation of | Result” >= |
| | | | hyperadrenocorticism. If additional assessment | (“Baseline |
| | | | for hyperadrenocorticism is indicated, consider | Result”* 0.5)) |
| | | | performing diagnostic imaging of the adrenal | And “Clinical |
| | | | glands such as abdominal ultrasound, computed | Signs” = |
| | | | tomography (CT) scan, or magnetic resonance | “No” |
| | | | imaging (MRI). | |
| | | | Hyperadrenocorticism is considered a clinical | |
| | | | syndrome. If the patient does not have clinical | |
| | | | signs consistent with | |
| | | | hyperadrenocorticism, treatment is not typically | |
| | | | recommended. | |
| | | | It is important to consider that administration of | |
| | | | exogenous steroids or stress related to concurrent | |
| | | | illness may affect the results and interpretation of | |
| | | | the dexamethasone suppression test. | |
| 1-1.5 | 1-1.5 | YES | The result of the low dose dexamethasone | If (“8 Hours |
| AND | AND | | suppression (LDDS) test in this dog | Result” >=1 |
| ≥50% | <50% | | is inconclusive and does not rule out a diagnosis | And “8 Hours |
| | | | of hyperadrenocorticism. | Result” <=1.5) |
| | | | In a dog with clinical signs consistent with | And “8 Hours |
| | | | hyperadrenocorticism, it is recommended to rule | Result” >= |
| | | | out non-adrenal causes for these clinical signs. If | (“Baseline |
| | | | an alternate cause for the clinical signs is not | Result”* 0.5) |
| | | | identified, additional testing for | And (“4 Hours |
| | | | hyperadrenocorticism should be considered, | Result” >=1 |
| | | | including an ACTH Stimulation test and | And “4 Hours |
| | | | abdominal ultrasound. | Result” <=1.5) |
| | | | | And (“4 Hours |
| | | | | Result” < |
| | | | | (“Baseline |
| | | | | Result”* 0.5)) |
| | | | | And “Clinical |
| | | | | Signs” = |
| | | | | “Yes” |
| 1-1.5 | 1-1.5 | NO | The result of the low dose dexamethasone | If (“8 Hours |
| AND | AND | | suppression (LDDS) test in this dog | Result” >=1 |
| ≥50% | <50% | | is inconclusive and does not rule out a diagnosis | And “8 Hours |
| | | | of hyperadrenocorticism. | Result” <=1.5) |
| | | | In a dog without clinical signs consistent with | And “8 Hours |
| | | | hyperadrenocorticism, no further testing for this | Result” >= |
| | | | disease is necessary at this time. In a dog with | (“Baseline |
| | | | abnormal laboratory results, it is recommended | Result”* 0.5) |
| | | | to rule out alternate causes for these | And (“4 Hours |
| | | | abnormalities. | Result” >=1 |
| | | | | And “4 Hours |
| | | | | Result” <=1.5) |
| | | | | And (“4 Hours |
| | | | | Result” < |
| | | | | (“Baseline |
| | | | | Result”* 0.5)) |
| | | | | And “Clinical |
| | | | | Signs” = |
| | | | | “No” |
| 1-1.5 | 1-1.5 | YES | The result of the low dose dexamethasone | If (“8 Hours |
| AND | AND | | suppression (LDDS) test in this dog | Result” >=1 |
| ≥50% | ≥50% | | is inconclusive and does not rule out a diagnosis | And “8 Hours |
| | | | of hyperadrenocorticism. | Result” <=1.5) |
| | | | In a dog with clinical signs consistent with | And “8 Hours |
| | | | hyperadrenocorticism, it is recommended to rule | Result” >= |
| | | | out non-adrenal causes for these clinical signs. If | (“Baseline |
| | | | an alternate cause for the clinical signs is not | Result”* 0.5) |
| | | | identified, additional testing for | And (“4 Hours |
| | | | hyperadrenocorticism should be considered, | Result” >=1 |
| | | | including an ACTH Stimulation test and | And “4 Hours |
| | | | abdominal ultrasound. | Result” <=1.5) |
| | | | | And (“4 Hours |
| | | | | Result” >= |
| | | | | (“Baseline |
| | | | | Result”* 0.5)) |
| | | | | And “Clinical |
| | | | | Signs” = |
| | | | | “Yes” |
| 1-1.5 | 1-1.5 | NO | The result of the low dose dexamethasone | If (“8 Hours |
| AND | AND | | suppression (LDDS) test in this dog | Result” >=1 |
| ≥50% | ≥50% | | is inconclusive and does not rule out a diagnosis | And “8 Hours |
| | | | of hyperadrenocorticism. | Result” <=1.5) |
| | | | In a dog without clinical signs consistent with | And “8 Hours |
| | | | hyperadrenocorticism, no further testing for this | Result” >= |
| | | | disease is necessary at this time. In a dog with | (“Baseline |
| | | | abnormal laboratory results, it is recommended | Result”* 0.5) |
| | | | to rule out alternate causes for these | And (“4 Hours |
| | | | abnormalities. | Result” >=1 |
| | | | | And “4 Hours |
| | | | | Result” <=1.5) |
| | | | | And (“4 Hours |
| | | | | Result” >= |
| | | | | (“Baseline |
| | | | | Result”* 0.5)) |
| | | | | And “Clinical |
| | | | | Signs” = |
| | | | | “No” |
| 1-1.5 | >1.5 | YES | The result of the low dose dexamethasone | If (“8 Hours |
| AND | | | suppression (LDDS) test in this dog | Result” >=1 |
| ≥50% | | | is inconclusive and does not rule out a diagnosis | And “8 Hours |
| | | | of hyperadrenocorticism. | Result” <=1.5) |
| | | | In a dog with clinical signs consistent with | And “8 Hours |
| | | | hyperadrenocorticism, it is recommended to rule | Result” >= |
| | | | out non-adrenal causes for these clinical signs. If | (“Baseline |
| | | | an alternate cause for the clinical signs is not | Result”* 0.5) |
| | | | identified, additional testing for | And “4 Hours |
| | | | hyperadrenocorticism should be considered, | Result” >1.5 |
| | | | including an ACTH Stimulation test and | And “Clinical |
| | | | abdominal ultrasound. | Signs” = |
| | | | | “Yes” |
| 1-1.5 | >1.5 | NO | The result of the low dose dexamethasone | If (“8 Hours |
| AND | | | suppression (LDDS) test in this dog | Result” >=1 |
| ≥50% | | | is inconclusive and does not rule out a diagnosis | And “8 Hours |
| | | | of hyperadrenocorticism. | Result” <=1.5) |
| | | | In a dog without clinical signs consistent with | And “8 Hours |
| | | | hyperadrenocorticism, no further testing for this | Result” >= |
| | | | disease is necessary at this time. In a dog with | (“Baseline |
| | | | abnormal laboratory results, it is recommended | Result”* 0.5) |
| | | | to rule out alternate causes for these | And “4 Hours |
| | | | abnormalities. | Result” >1.5 |
| | | | | And “Clinical |
| | | | | Signs” = |
| | | | | “No” |
| — | — | YES/NO | For anything else - | |
| | | | No result. Please check values. | |

By reference to the predetermined rule set n the tables, the clinical interpretation (shown under the column “Text”) can be selected using the algorithm shown.

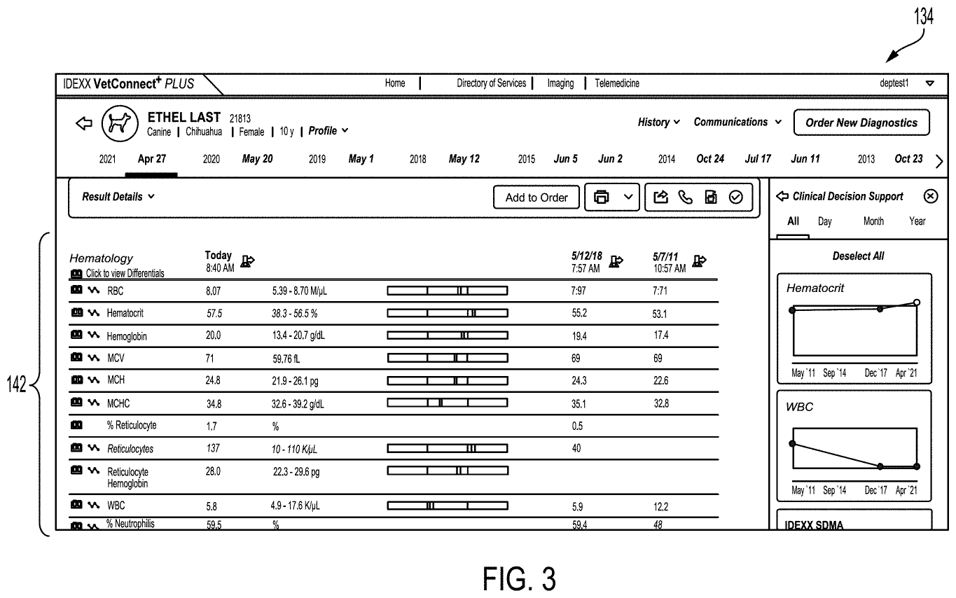

Referring to FIGS. 3-7, the graphical user interface 134, in embodiments, is a user interface that allows users to interact with the computing device 102 to provide inputs, for example, through displaying graphical icons and/or results.

The clinical decision support interface 136 is a component of the graphical user interface 134 and can be displayed as a window or an overlay in the graphical user interface 134 to provide information in an organized manner.

The instructions 124, in some embodiments, includes a recommendation module 138. The recommendation module 124 is executed to identify and determine appropriate recommendations for follow-on testing to provide based on any of a number of factors including but not limited to, the test results, historical test results, test results observed by other veterinary laboratories with patients in similar circumstances, and the like. In some embodiments, “recommendations” may comprise a list of testing options presented to the user.

In some embodiments, the instructions 124 includes a machine learning algorithm. The machine learning algorithm 140 uses statistical models to generate the recommendation of follow-on testing to be performed. The machine learning algorithm 140 can generate the recommendation of follow-on testing effectively without using explicit instructions, but instead, by relying on patterns and inferences. In one example, the computing device 102 (FIG. 1) receives outputs of diagnostic tests performed by the diagnostic testing instruments 104a-n (FIG. 1) positioned in the plurality of veterinary laboratories 118a-n (FIG. 1) by accessing the lab test results database 120 (FIG. 1). The computing device 102 (FIG. 1) uses the machine learning algorithm 140 (FIG. 2) to process the outputs of diagnostic tests performed by diagnostic testing instruments 104a-104n (FIG. 1) positioned in the plurality of veterinary laboratories 118a-n (FIG. 1) so as to identify patterns of outputs and associated follow-on testing performed. In embodiments, the computing device 102 (FIG. 1) then generates the recommendation of follow-on testing to perform based at least in part on the identified patterns of outputs and associated follow-on testing performed at the plurality of veterinary laboratories 118a-n (FIG. 1).

The machine learning algorithm 140 can utilize data in the lab test results database 120 as a knowledge base of training data to learn of symptoms and test results for which certain follow-on testing was performed. The machine learning algorithm 140 can also utilize data in the lab test results database 120 as a knowledge base of training data to learn if the follow-on testing was successful, such as a comparison of test result data over time to determine whether a condition has improved.

Within one example, in operation, when the instructions 124 are executed by the one or more processor(s) 122, the one or more processor(s) 122 are caused to perform functions including receiving a diagnostic test result for an animal patient as a result of a series of diagnostic tests performed on the animal patient, based on the diagnostic test result being indicative of a steroid analyte, initiating an automated clinical decision support interface 136 on a graphical user interface 134 for the diagnostic test result for the animal patient, in response to receiving a selection on the graphical user interface 134 to initiate automated clinical decision support for the diagnostic test result for the animal patient, prompting a user via the graphical user interface 134 to provide input regarding (i) a dose of medication provided to the animal patient for the diagnostic test, and (ii) information relating to at least one observed clinical sign in the animal patient, based on (i) the dose of medication provided to the animal patient and (ii) the information relating to the at least one observed clinical sign in the animal patient, executing a set of predetermined rules 132 for processing the diagnostic test result for the animal patient to generate a clinical interpretation of the diagnostic test, and responsively providing via the graphical user interface 134 the clinical interpretation of the diagnostic test.

Thus, the instructions 124 are executable for providing assistance in a form of automated clinical decision-making for a veterinarian or laboratory technician to further create an efficient workflow process in the location 116, for example.

FIG. 3 is an example illustration of the graphical user interface 134 illustrating diagnostic test results, according to an example implementation. In one example, the computing device 102 provides for display, the graphical user interface 134 including a representation 142 of the diagnostic test result for the series of diagnostic tests performed on the animal patient in rows and columns. For example, when the diagnostic test includes dexamethasone suppression testing, such testing requires running at least two tests and as many as five tests on the animal patient, and results of the tests for different dates can be shown in different columns. Each analyte for which a blood analysis is performed can be shown in a different row, for example.

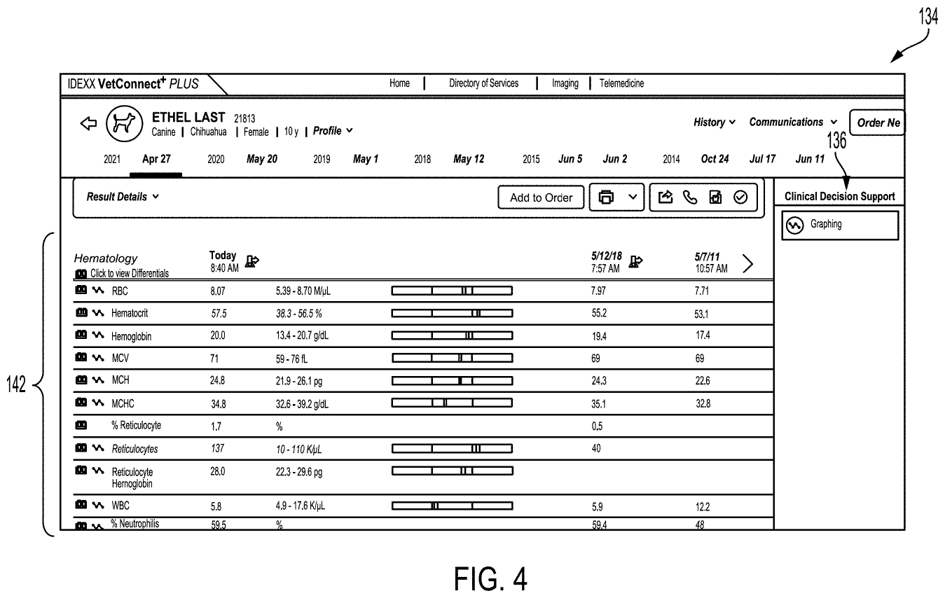

FIG. 4 is an example illustration of the graphical user interface 134 illustrating diagnostic test results with the clinical decision support interface 136, according to an example implementation. Upon display of the graphical user interface 134, the computing device 102 programmatically initiates the automated clinical decision support interface 136 on the graphical user interface 134 for the diagnostic test result for the animal patient. This includes providing for display a side panel on the graphical user interface 136 to prompt the user to provide input(s), and the side panel overlays at least a portion of the representation of the diagnostic test result.

As such, users will have an option to engage with the graphical user interface 134 to receive further information on interpretation of the dexamethasone suppression test, for example, through use of the clinical decision support interface 136.

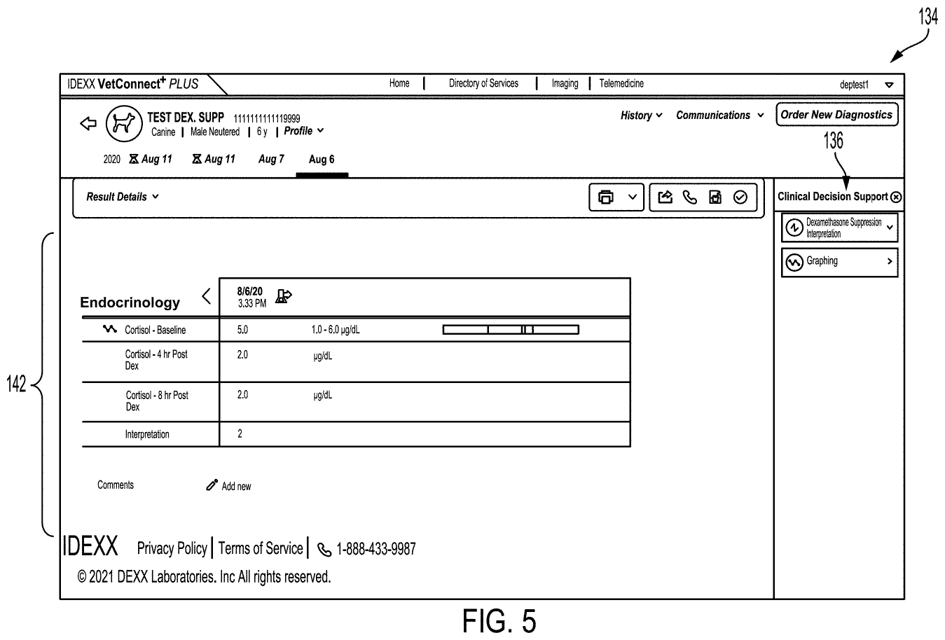

FIG. 5 is an example illustration of the graphical user interface 134 illustrating diagnostic test results with the clinical decision support interface 136 offering more selections for interpretation, according to an example implementation. In FIG. 5, the clinical decision support interface 136 includes a selection for “Dexamethasone Suppression Interpretation,” for example.

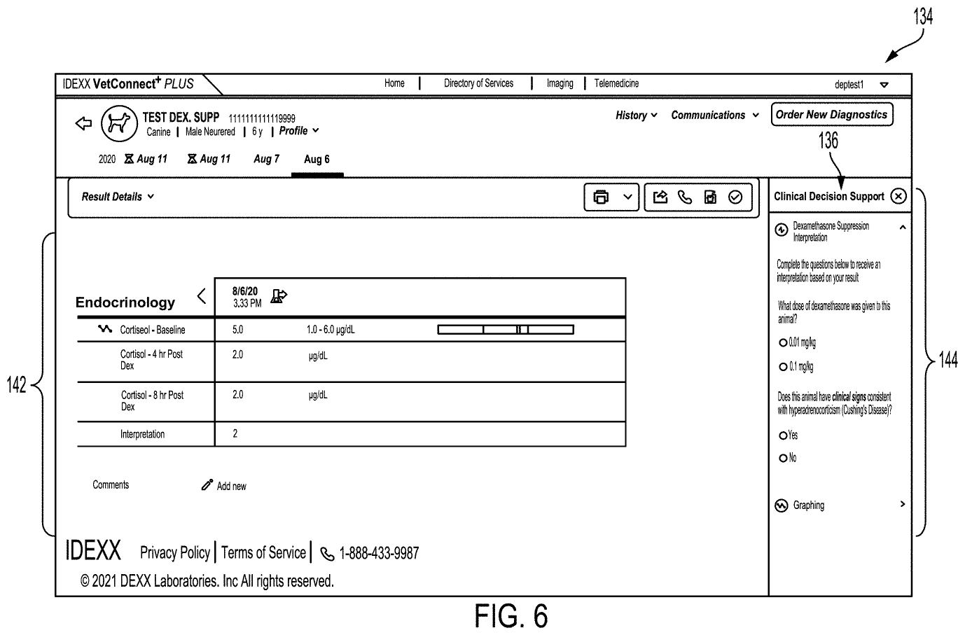

FIG. 6 is another example illustration of the graphical user interface 134 illustrating diagnostic test results with the clinical decision support interface 136, according to an example implementation. In FIG. 6, a user selected “Dexamethasone Suppression Interpretation” in the clinical decision support interface 136, and prompts 144 for input are displayed including a prompt for (i) a dose of medication provided to the animal patient for the diagnostic test, and (ii) information relating to at least one observed clinical sign in the animal patient. The prompts are shown as buttons for selection; however, the prompts may additionally or alternatively include fields into which a user may type information.

The prompts 144 are required here for Dexamethasone Suppression Interpretation because the set of predetermined rules 132 require such inputs requested by the prompts 144 for execution. For other clinical interpretation, alternative prompts may be generated. Thus, the computing device 102 may generate prompts for user input based on the diagnostic test performed, as well as, based on reference to the set of predetermined rules 132 so as to determine inputs required to execute the set of predetermined rules 132.

Following receipt of the input(s) into the clinical decision support interface 136, the computing device 102 executes the set of predetermined rules 132 for processing the diagnostic test result for the animal patient. For example, the computing device 102 (FIG. 1) accesses, within a database (e.g., the diagnostic testing rules database 106 (FIG. 1)), the set of clinical interpretations 112 of the diagnostic test associated with an amount of the dose of medication provided to the animal patient (e.g., as shown in Tables 1 and 2 above), and maps the diagnostic test result with one of the clinical interpretations in the set of clinical interpretations 112 based on the level of the hormone in the animal patient being in a range of the level of hormone associated with the one of the clinical interpretations. Such mapping also takes into account the input received on the clinical decision support interface 136 including an indication of high/low dose and indication of clinical signs of the patient.

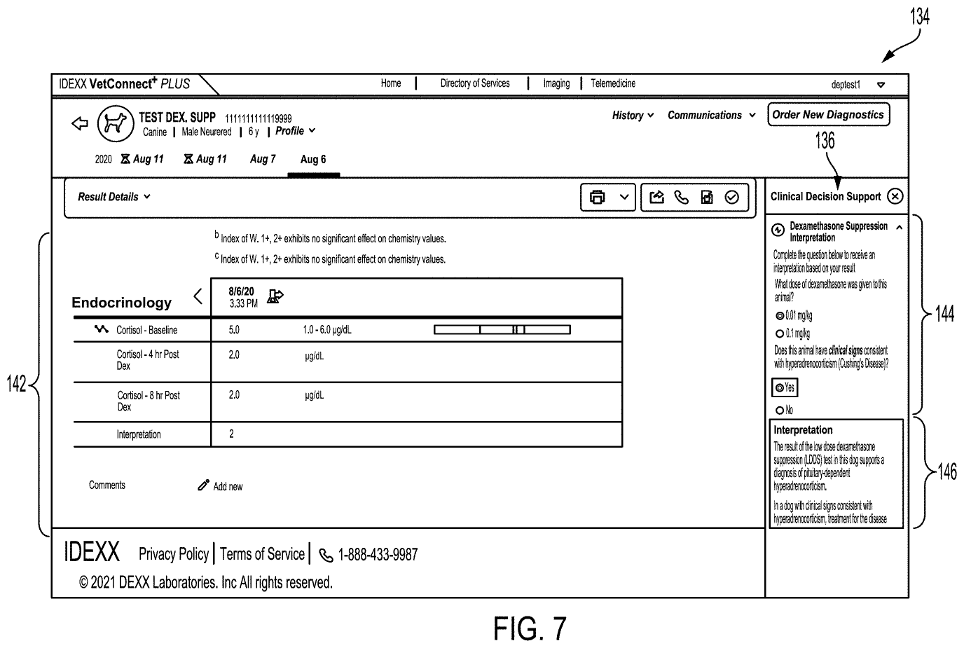

After mapping the diagnostic test result with one of the clinical interpretations in the set of clinical interpretations 112, the computing device 102 provides the clinical interpretation for display in the clinical decision support interface 136. FIG. 7 is another example illustration of the graphical user interface 134 illustrating diagnostic test results with the clinical decision support interface 136 illustrating the clinical interpretation 146, according to an example implementation.

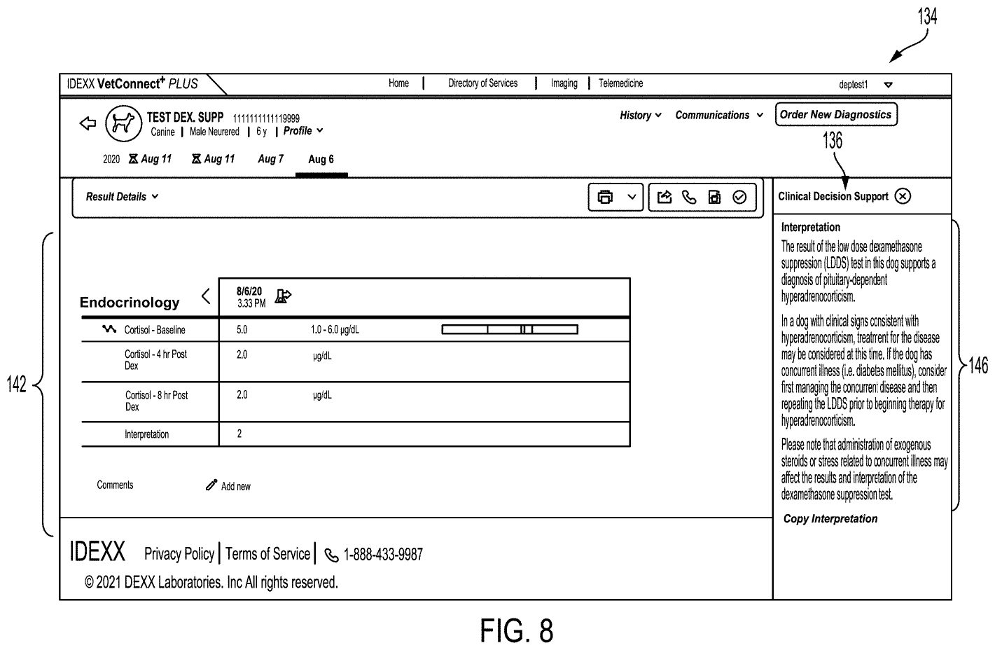

FIG. 8 is another example illustration of the graphical user interface 134 illustrating diagnostic test results with the clinical decision support interface 136 illustrating the clinical interpretation 146, according to an example implementation. In FIG. 8, the prompts 144 are not shown. The examples shown in FIGS. 7-8 illustrate the clinical interpretation 146 being that the test does not support a diagnosis of hyperadrenocorticism.

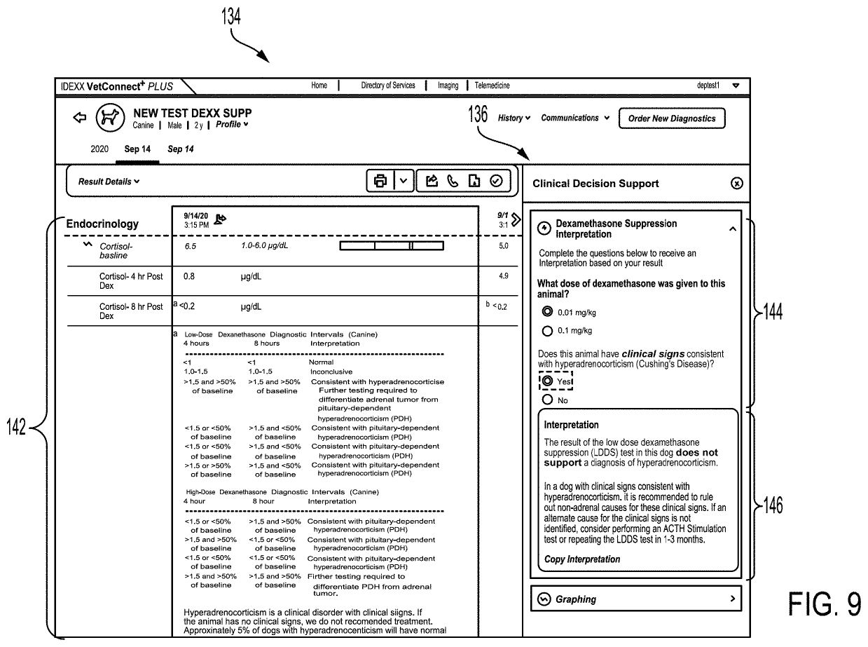

FIG. 9 is another example illustration of the graphical user interface 134 illustrating diagnostic test results with the clinical decision support interface 136 illustrating the clinical interpretation 146, according to an example implementation. In FIG. 9, the example illustrates that the test support a diagnosis of pituitary-dependent hyperadrenocorticism.

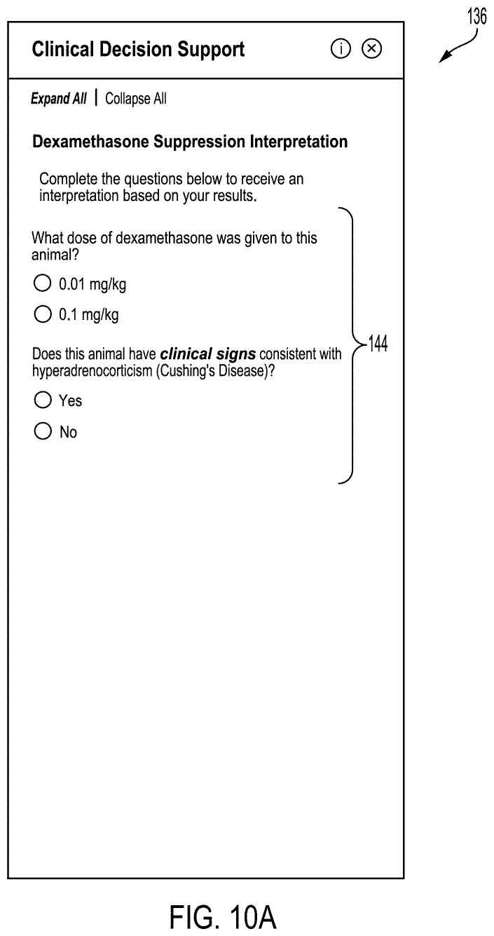

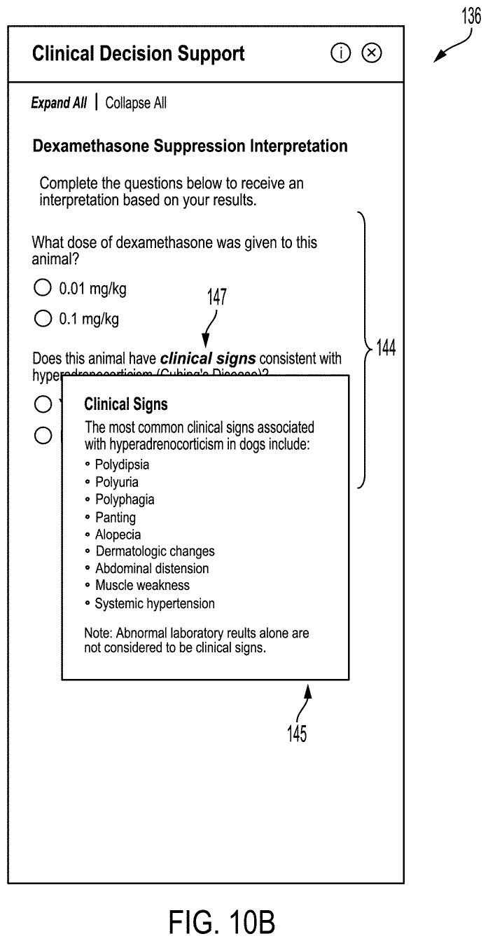

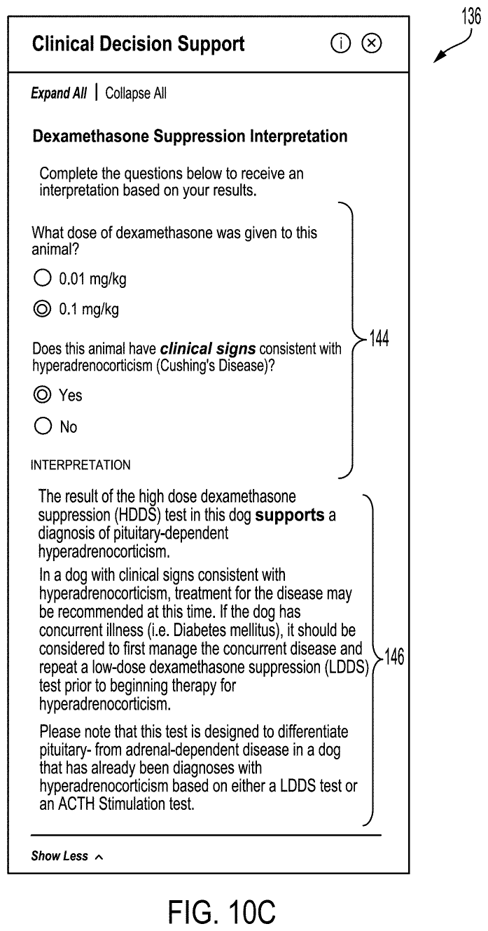

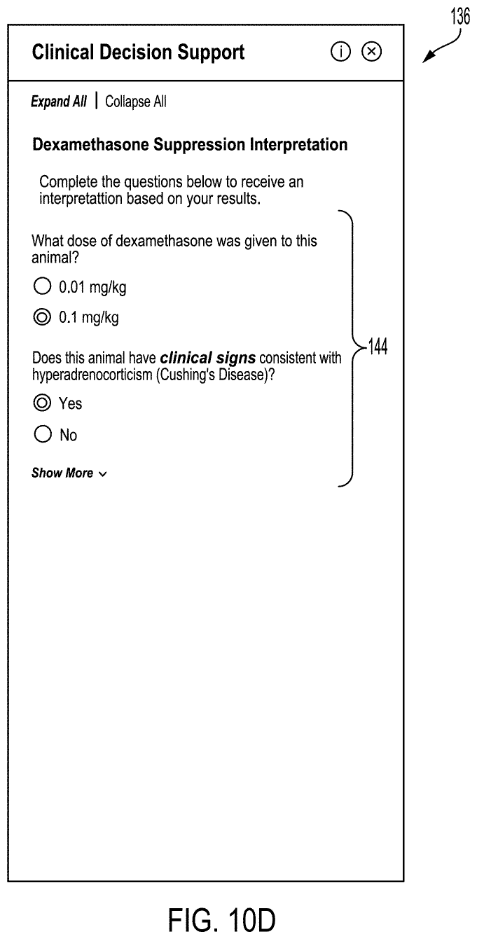

FIGS. 10A-10D illustrate examples of the clinical decision support interface 136 with details for the Dexamethasone Suppression Interpretation, according to example implementations. In FIG. 10A, the clinical decision support interface 136 includes the Dexamethasone suppression interpretation and the prompts 144. In FIG. 10B, the clinical decision support interface 136 is illustrated with a pop-up graphic 145 that is triggered for display based on a mouse-over input on a hyperlink 147. The hyperlink 147 in FIG. 10B of clinical signs thus causes the pop-up graphic 145 to be displayed including details of the clinical signs associated with a condition being analyzed (e.g., here a condition of hyperadrenocorticism in dogs has associated signs of polydipsia, polyuria, polyphagia, panting, alopecia, dermatologic changes, abdominal distension, muscle weakness, and systemic hypertension). Abnormal laboratory results alone are not considered clinical signs, in some examples. In FIG. 10C, the clinical decision support interface 136 is illustrated with the prompts 144 and the clinical interpretation 146. In FIG. 10D, the clinical decision support interface 136 is illustrated without the clinical interpretation 146 as a user may select “show less” or “show more” on the clinical decision support interface 136 to display or hide the clinical interpretation.

FIGS. 10A-10D illustrate different components of the clinical decision support interface 136 including header, summary, hyperlink text, prompts, and/or clinical interpretation, each of which is triggered for display following receipt of input(s) into the clinical decision support interface 136 and/or via the computing device 102 executing the set of predetermined rules 132 for processing the diagnostic test result for the animal patient. As a result, the clinical decision support interface 136 has content for display generated dynamically per patient.

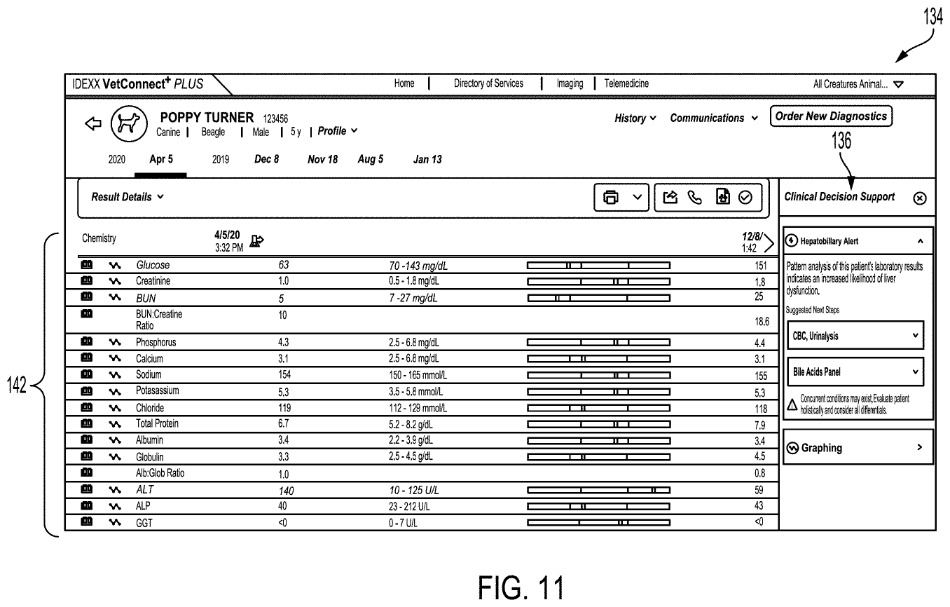

FIGS. 11-14 illustrate an example of the graphical user interface 134 illustrating diagnostic test results with the clinical decision support interface 136 with details for a hepatobiliary alert, according to an example implementation.

In FIG. 11, the clinical decision support interface 136 includes details for a hepatobiliary alert, for example. In this example, the computing device 102 receives the diagnostic test results for the animal patient, and generates the clinical decision support interface 136 for display on the graphical user interface 134 according to the diagnostic test results received. In addition, or alternatively, the computing device 102 generates the clinical decision support interface 136 for display on the graphical user interface 134 according to execution of the set of predetermined rules 132 for processing the diagnostic test result for the animal patient. In the example shown in FIG. 11, the diagnostic test results indicate an increased possibility of liver dysfunction, and based on execution of the set of predetermined rules 132, the computing device 102 programmatically generates the clinical decision support interface 136 to include a hepatobiliary alert for inclusion in the automated clinical decision support interface 136.

In one example, to determine the increased liver dysfunction, patterns in the diagnostic test results are identified anywhere from two to five chemistry analytes, and/or a urinalysis parameter, and/or a hematology parameter.

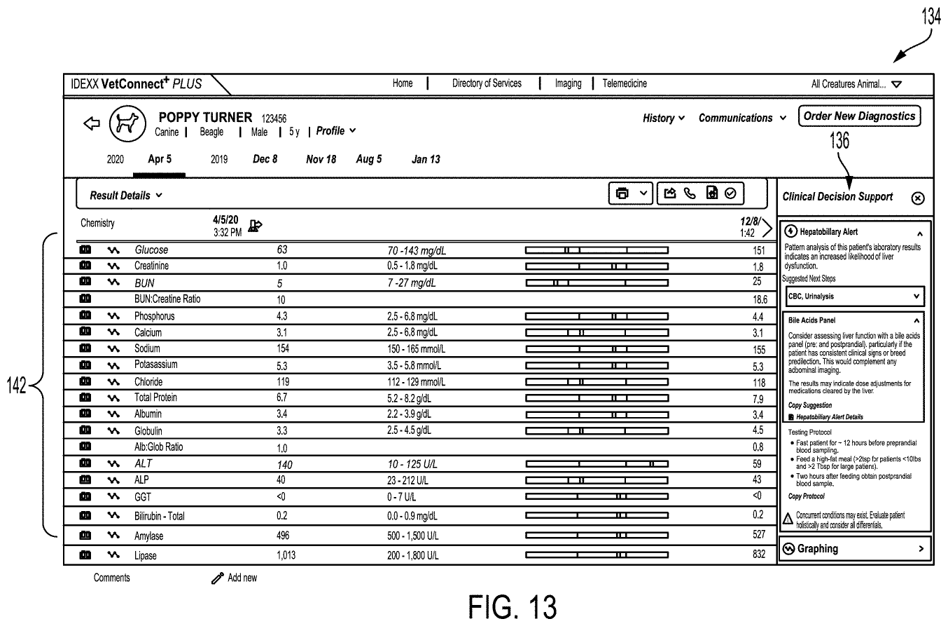

The hepatobiliary alert may include information relating to a complete blood count (CBC), urinalysis and a bile acids panel. To generate the hepatobiliary alert, the computing device 102 executes the set of predetermined rules, which includes dynamically generating CBC, urinalysis, and/or chemistry next step suggestions based on a lack of CBC, urinalysis, and chemistry test results from tests performed on the animal patient within about a past month timeframe. The CBC, urinalysis, and/or chemistry next step suggestions can be based on which elements of a minimum database (e.g., testing) have been run within the past 28 days, for example. If these tests have been run within the past month timeframe, the CBC, urinalysis, and/or chemistry next step suggestions may be omitted from the hepatobiliary alert. In some embodiments, the computing device 102 can be programmed, to execute the set of predetermined rules to include a bile acids panel suggestion based on the diagnostic test results being indicative of the increased possibility of liver dysfunction regardless of the presentation of CBC, urinalysis, and/or chemistry next step suggestions. The bile acids panel suggestion may, for example, include information related to a testing protocol for use to conduct a bile acids panel diagnostic test on the animal patient (shown in FIG. 13). Additional information, such as a caution note, is included as a reminder of limitations of the alert, in some examples.

In some embodiments, the computing device 102 executes the set of predetermined rules to create the hepatobiliary alert including the CBC, urinalysis, and/or chemistry next step suggestions as well as the bile acids panel suggestion for inclusion in the automated clinical decision support interface 136, and then publishes the hepatobiliary alert in the automated clinical decision support interface, as shown in FIG. 11.

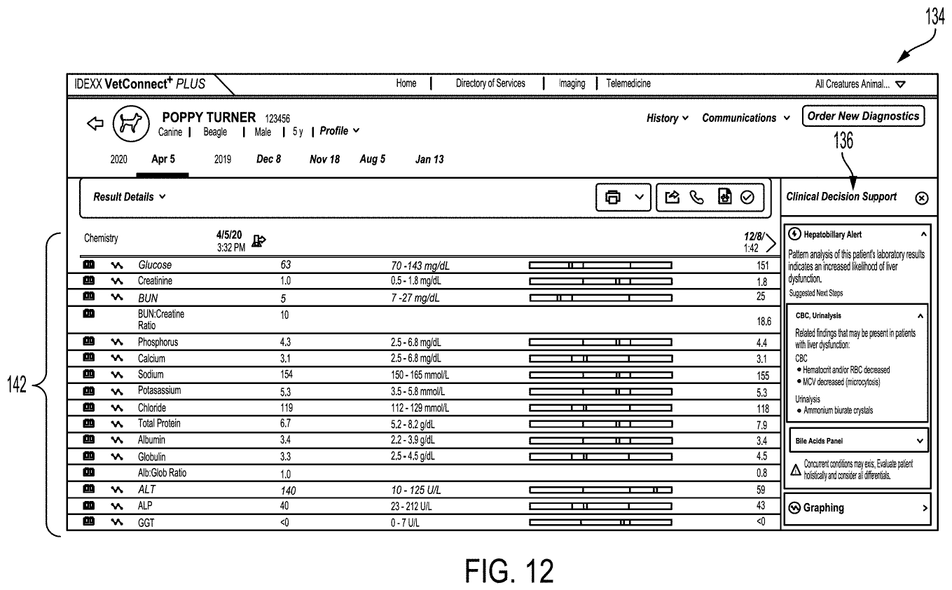

FIG. 12 is another example illustration of the graphical user interface 134 illustrating diagnostic test results with the clinical decision support interface 136, according to an example implementation. In FIG. 12, a user selected “CBC, Urinalysis” in the clinical decision support interface 136 (as shown in FIG. 11), and related findings for each test are populated dynamically so as to provide further information. In the example in FIG. 12, the computing device 102 references testing database to retrieve information based on selection of the CBC, Urinalysis drop-down menu and causes display of the information such as “Hematocrit and/or RBC decreased” and “MCV decreased (microcytosis)”, as well as “Ammonium biurate crystals”. Information is populated dynamically into the clinical decision support interface 136 by the computing device 102 referencing related databases as a result of execution of the set of predetermined rules 132.

FIG. 13 is another example illustration of the graphical user interface 134 illustrating diagnostic test results with the clinical decision support interface 136, according to an example implementation. In FIG. 13, a user selected “Bile Acids Panel” in the clinical decision support interface 136 (as shown in FIG. 11), and related findings for each test are populated dynamically so as to provide further information. In the example in FIG. 13, the computing device 102 references testing database to retrieve information based on selection of the Bile Acids Panel drop-down menu and causes display of the information such as additional explanation of the results, hyperlinks to hepatobiliary alert details (e.g., clickable by user to determine full bile acids algorithm and details on why the alert was generated), and testing protocols. Information is populated dynamically into the clinical decision support interface 136 by the computing device 102 referencing related databases as a result of execution of the set of predetermined rules 132.

The computing device 102 executes a bile acids (BA) algorithm to identify patterns in the diagnostic test results based on CBC, chemistry, and Urinalysis patterns associated with BA>30 micromole per liter (μmol/L), for example. The computing device 102 executes the set of predetermined rules 132 to identify patterns, such as a population of patients where a threshold number (e.g., 50%) of those tested with similar patterns indicative of liver dysfunction. An example bile acids algorithm initially considers information about the patient such as clinical signs of breed predilection, poor growth, poor recovery from anesthesia/sedation, neurologic signs, history of hepatotoxic medication, weight loss, anorexia/vomiting/diarrhea, ascites, and icterus. The computing device 102 then analyzes the diagnostic test results to determine decreased CBC, decreased or low chemistry panel data, and/or anomalies in urinalysis. Following, the computing device 102 receives the information about the patient, as well as the diagnostic test results (e.g., CBC, chemistry panel, and/or urinalysis), and based on two or more clinical indicators from the information about the patient and the diagnostic test result being present, further decisions are carried out as a clinical support tool identifying that the patient is “normal,” experiencing “mild elevation,” or experiencing “moderate to severe elevation”.

Thus, the computing device 102 executes the set of predetermined rules 132 for clinical decision support resulting in the hepatobiliary alert in the graphical user interface 134, as shown in FIGS. 11-14. The computing device 102 receives the diagnostic test results, and based on what test results are received, a customized clinical decision support interface 136 is generated for display. The computing device 102 maps the diagnostic test result with one of the clinical interpretations in the set of clinical interpretations 112 based on testing analyzed. For the hepatobiliary alert, testing of the C-reactive protein (CRP) is utilized to characterize severity of inflammation in the patient, and in combination with the CBC, is utilized by the computing device 102 to make associated hepatobiliary alerts.

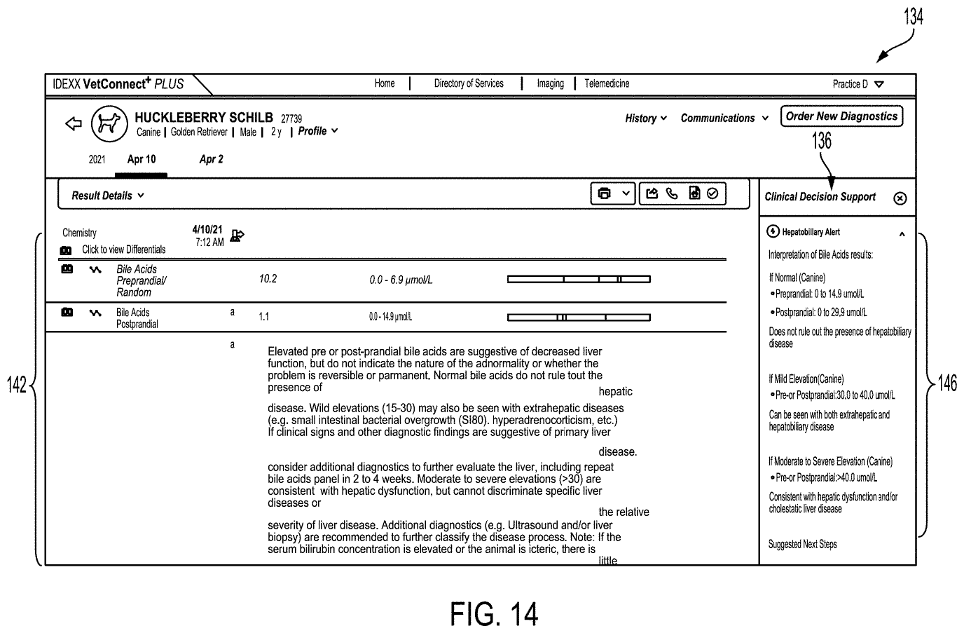

After mapping the diagnostic test result with one of the clinical interpretations in the set of clinical interpretations 112, the computing device 102 provides the clinical interpretation for display in the clinical decision support interface 136. FIG. 14 is another example illustration of the graphical user interface 134 illustrating diagnostic test results with the clinical decision support interface 136 illustrating the clinical interpretation 146, according to an example implementation. As shown in FIG. 13, interpretation of the bile acids results are illustrated for display in the clinical decision support interface 136. In the example shown in FIG. 14, the interpretation indicates normal results for the patient, and such interpretation is dynamically generated (in real-time as the user provides selection of the drop-down menus). “Real-time” includes execution of the predetermined rules by the computing device 102 as user inputs are received, or within a response time having a preset maximum limit or constraint.

Thus, as shown in FIG. 14, the computing device 102 executes the set of predetermined rules to responsively provide, via the graphical user interface 134, a clinical interpretation of the bile acids panel diagnostic test results based on receipt of such test results.

In some examples, the level of the hormone in the animal patient is outside of the range of the level of hormone associated with any of the clinical interpretations in the set of clinical interpretations 112. In this example, the computing device 102 may require more information to generate the clinical interpretation. As a result, the computing device 102 may be programmed to access patient test records for the animal patient within the patient information database 110, and generate the clinical interpretation of the diagnostic test by reference to the patient test records for the animal patient. In some examples, the computing device 102 can indicate that the data is inconclusive.

In further examples, the computing device 102 executes the set of predetermined rules 132 for processing the diagnostic test result for the animal patient by further accessing the patient information database 110 to receive one or more characteristics of the animal patient selected from the group including, for example and without limitation, species, weight, age, and/or the like, and then generates the clinical interpretation of the diagnostic test based on the one or more characteristics of the animal patient. By receiving the characteristics of the patient, the computing device 102 has information useful to filter out possible clinical interpretations from clinical interpretations stored in the memory (e.g., the clinical interpretations 112) based on the characteristics of the animal patient, such as to access in interpretations applicable to a certain breed, for example.

In some examples, the computing device 102 is further programmed to generate a recommendation for treatment or additional testing based on the clinical interpretation, and responsively provide via the graphical user interface 134 the recommendation.

The recommendation can be generated based on a number of factors including the output of the diagnostic test and historical test results of the patient. In this regard, referring back to FIG. 1, the system 100 includes the patient information database 110 (or Practice Information Management Software “PIMS” database) that stores and manages information related to a patient. Such information can include name, date of birth, address, sex, breed, and associated medical data (e.g., blood chemistry test results, hematology test results, infectious disease test results, non-infectious disease test results, urinalysis test results, cytology data, morphology data, radiology images, immunoassay test result images, and billing data etc.). The computing device 102 can access the patient information database 112 to retrieve historical test results of the patient, and compare the historical test results to the current diagnostic test result so as to make a recommendation of any follow-up or follow-on testing that should be performed.

The computing device 102 can receive outputs of a plurality of diagnostic tests performed by the plurality of diagnostic testing instruments 104a-n (or by any number of the diagnostic testing instruments 104a-n), and then generate the recommendation of the follow-on testing to perform based on all outputs received from any and all of the diagnostic tests. In this way, the computing device 102 utilizes all available information to make recommendations of further testing to perform.

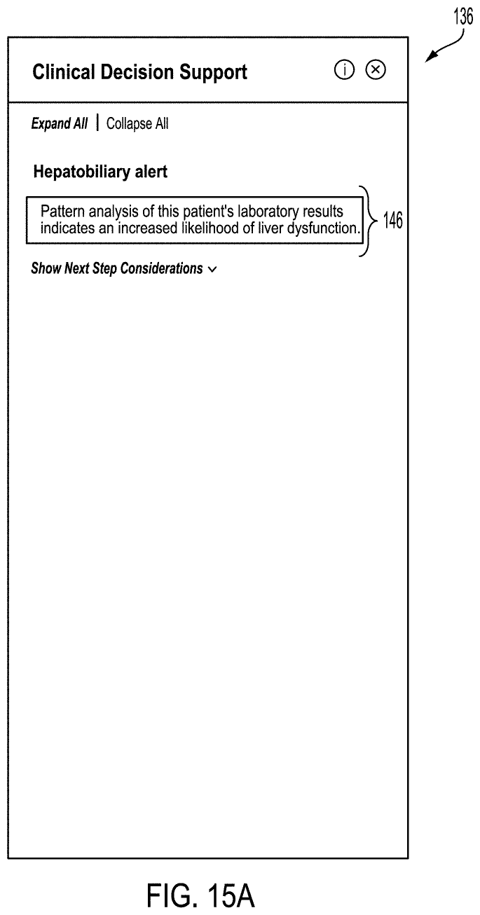

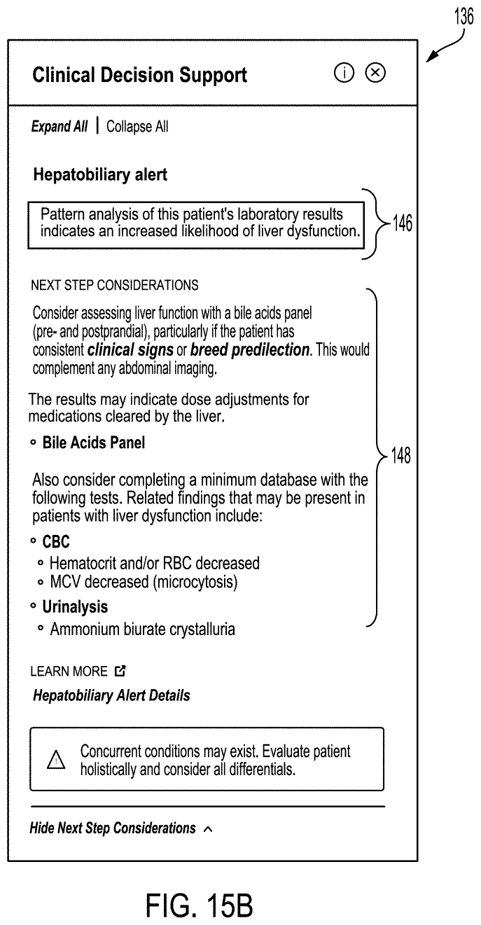

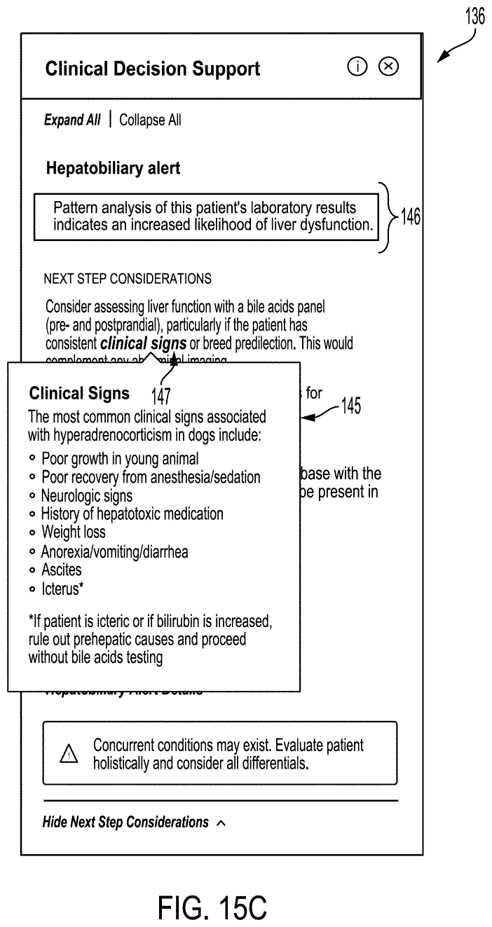

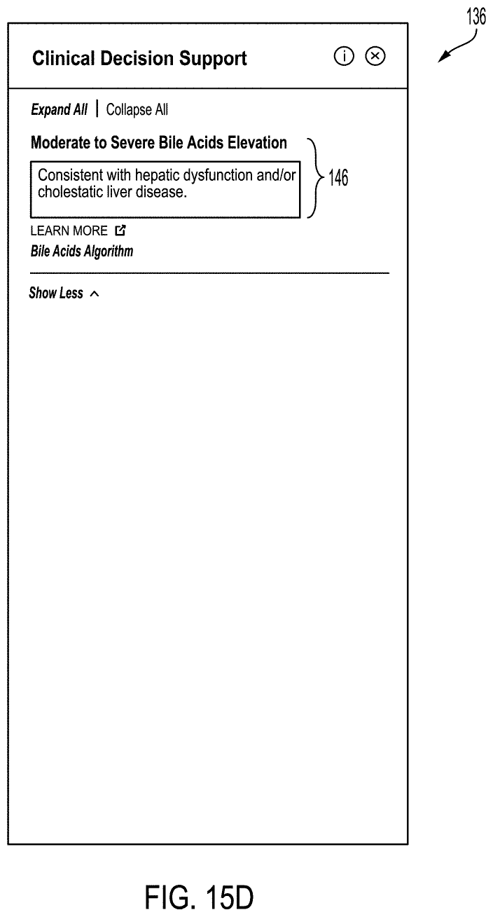

FIGS. 15A-15D illustrate examples of the clinical decision support interface 136 with details for the hepatobiliary alert, according to example implementations. In FIG. 15A, the clinical decision support interface 136 includes the clinical interpretation 146 for the hepatobiliary alert. In FIG. 15B, the clinical decision support interface 136 is illustrated with next step considerations 148 including data for follow-on tests to perform. The follow-on tests are other diagnostic testing recommended to perform based on the hepatobiliary alert being triggered. Upon selection of a follow-on test, such as bile acids panel, CBC, or urinalysis as shown in FIG. 15B, the computing device 102 accesses associated test codes, populates patient information, and enables a user to place an order for the test, for example. In FIG. 15C, the clinical decision support interface 136 is illustrated with the pop-up graphic 145 that is triggered for display based on a mouse-over input on the hyperlink 147. The hyperlink 147 in FIG. 15C of clinical signs thus causes the pop-up graphic 145 to be displayed including details of the clinical signs associated with a condition being analyzed (e.g., here a condition of hyperadrenocorticism in dogs has associated signs of poor growth in young animal, poor recovery from anesthesia/sedation, neurologic signs, history of hepatotoxic medication, weight loss, anorexia/vomiting/diarrhea, ascites, and icterus). In FIG. 15D, the clinical decision support interface 136 is illustrated with the clinical interpretation 146 and a hyperlink text for the Bile Acids Algorithm in an instance where selection of Bile Acids Panel in the next step considerations 148 is selected.

FIGS. 15A-15D illustrate different additional components of the clinical decision support interface 136 including header, summary, hyperlink text, prompts, and/or clinical interpretation, each of which is triggered for display following receipt of input(s) into the clinical decision support interface 136 and/or via the computing device 102 executing the set of predetermined rules 132 for processing the diagnostic test result for the animal patient. As a result, the clinical decision support interface 136 has content for display generated dynamically per patient.

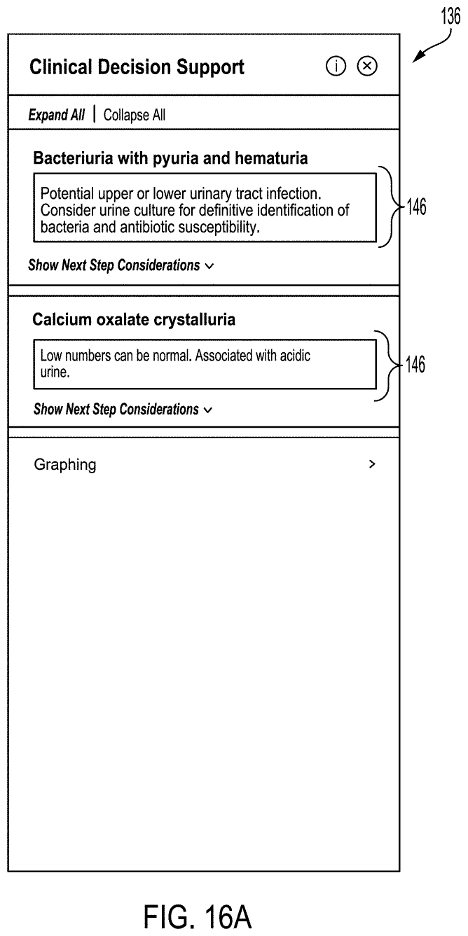

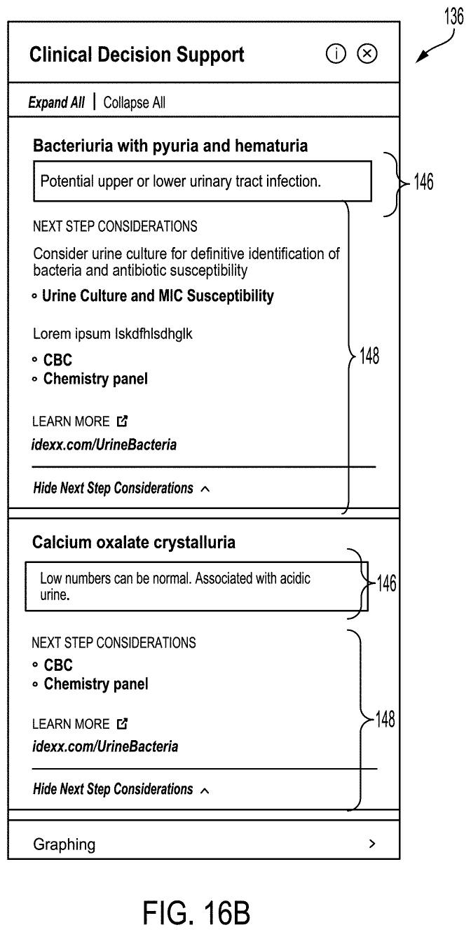

FIGS. 16A-16B illustrate examples of the clinical decision support interface 136 with details for the urinalysis test, according to example implementations. In FIG. 16A, the clinical decision support interface 136 includes the clinical interpretation 146 for the urinalysis. In FIG. 16B, the clinical decision support interface 136 is illustrated with next step considerations 148 including data for follow-on tests to perform. The follow-on tests are other diagnostic testing recommended to perform based on the clinical interpretation 146 indicating a potential upper or lower urinary tract infection, for example. Upon selection of a follow-on test, such as urine culture, CBC, or chemistry panel as shown in FIG. 15B, the computing device 102 accesses associated test codes, populates patient information, and enables a user to place an order for the test, for example. In FIG. 16B, the clinical decision support interface 136 is also illustrated with the clinical interpretation 146 for calcium oxalate crystalluria analysis, and associated next step considerations 148.

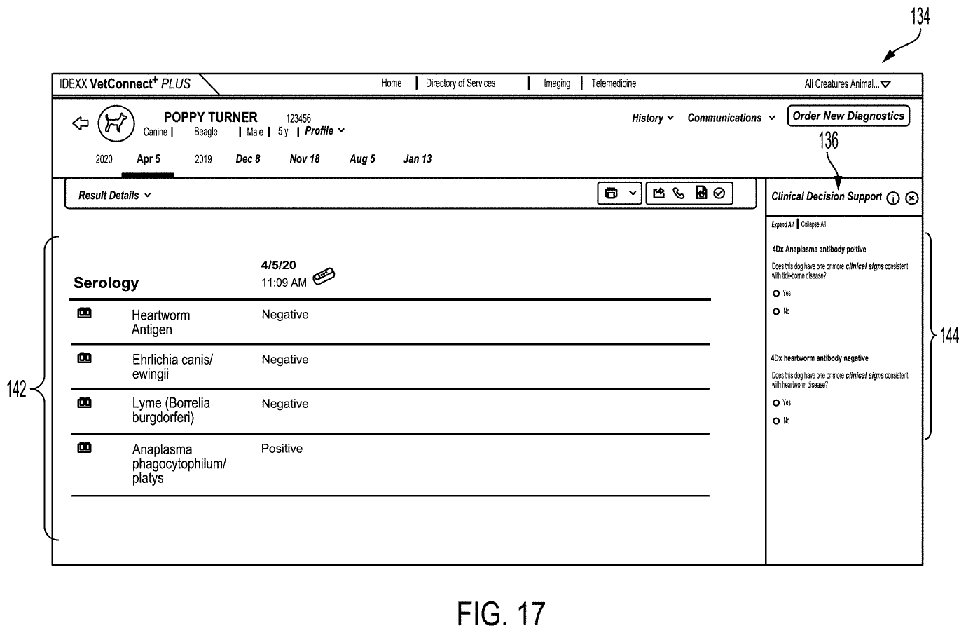

FIG. 17 is an example illustration of the graphical user interface 134 illustrating still other diagnostic test results with the clinical decision support interface 136, according to an example implementation. In FIG. 17, the clinical decision support interface 136 includes a selection for “4Dx Anaplasma antibody positive” and “4Dx heartworm antigen negative,” for example. The “4Dx” test refers to a blood test that checks for four common diseases in dogs: Heartworm, plus three ick-borne diseases. The 4Dx test is a screening test offering a yes (positive) or no (negative) result.

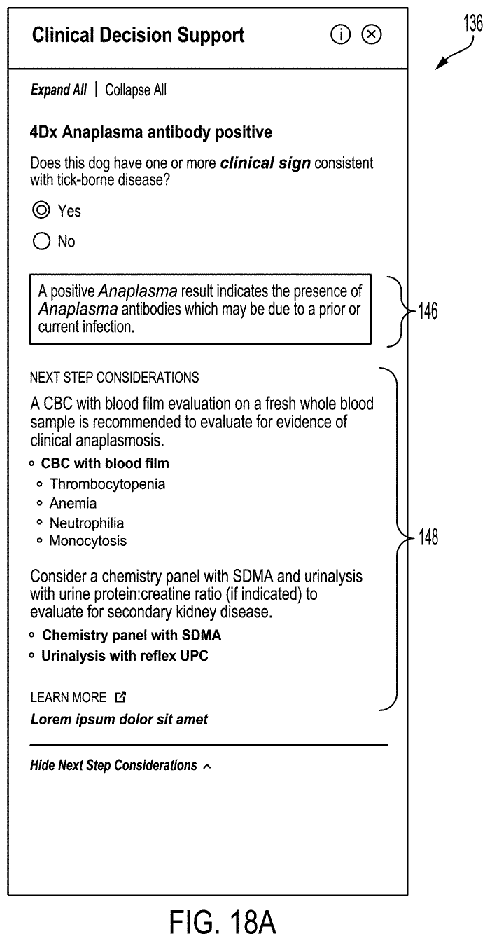

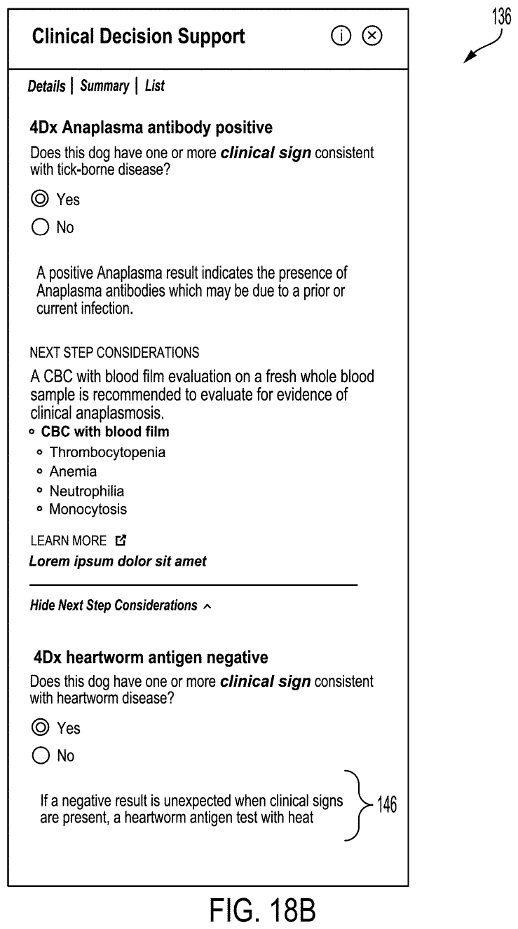

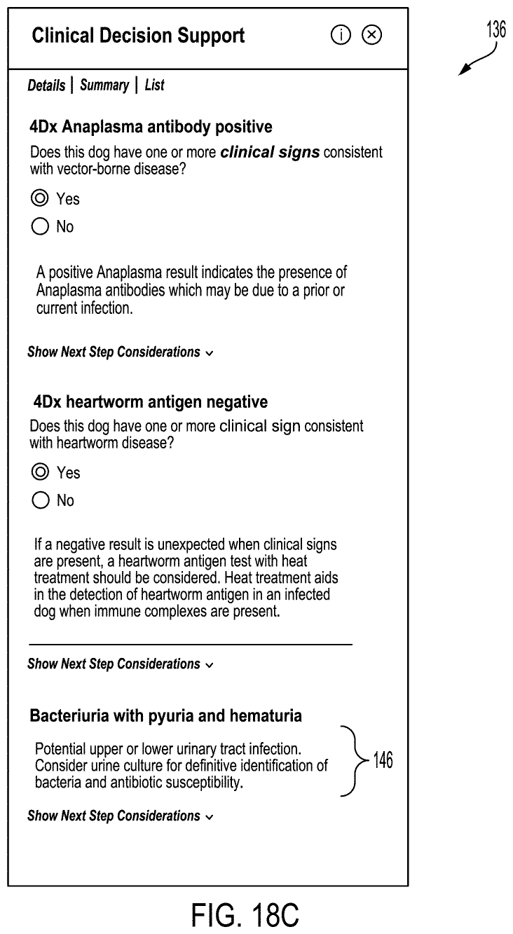

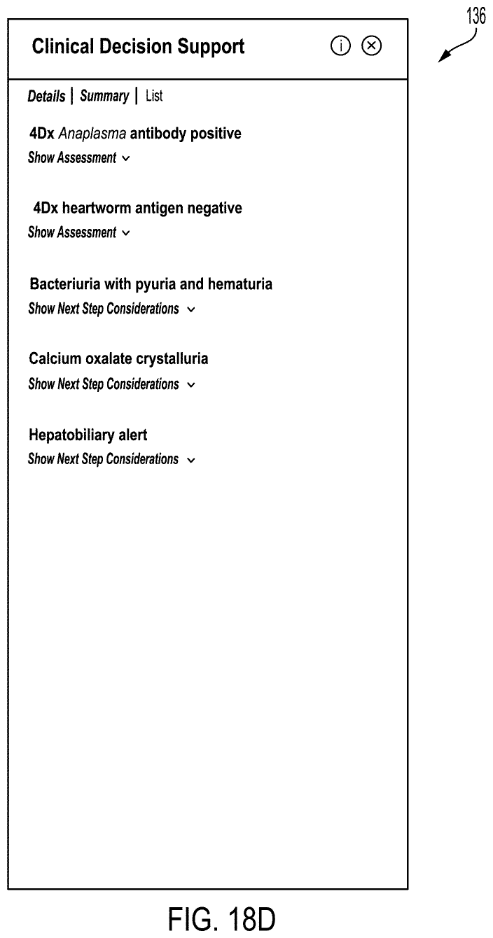

FIGS. 18A-18D illustrate examples of the clinical decision support interface 136 with details for the 4Dx alert (in FIG. 17), according to example implementations. In FIG. 18A, an example of the clinical decision support interface 136 with details for the 4Dx anaplasma antibody test, according to example implementations. Thus, upon input of yes in the clinical decision support interface in FIG. 18A, the clinical decision support interface 136 dynamically updates display with new information including the clinical interpretation 146 and the next step considerations 148 for the identified condition of positive. Example follow-on testing includes CBC with blood film, chemistry panel, and urinalysis. In FIG. 18B, the clinical decision support interface 136 is illustrated with an input for 4Dx heartworm antigen negative and the clinical interpretation 146. In FIG. 18C, the clinical decision support interface 136 is illustrated with additional input for the clinical interpretation 146 including bacteriuria with pyuria and hematuria. In FIG. 18D, various conditions described herein are illustrated in an example in which a patient has been tested for each of the conditions. The conditions are all illustrated in a collapsed view where assessments (the clinical interpretation 146) and the next step considerations 148 are accessible by selection of the hyperlink text. Thus, FIG. 18D illustrates a compact view and graphical display of the clinical decision support interface 136.

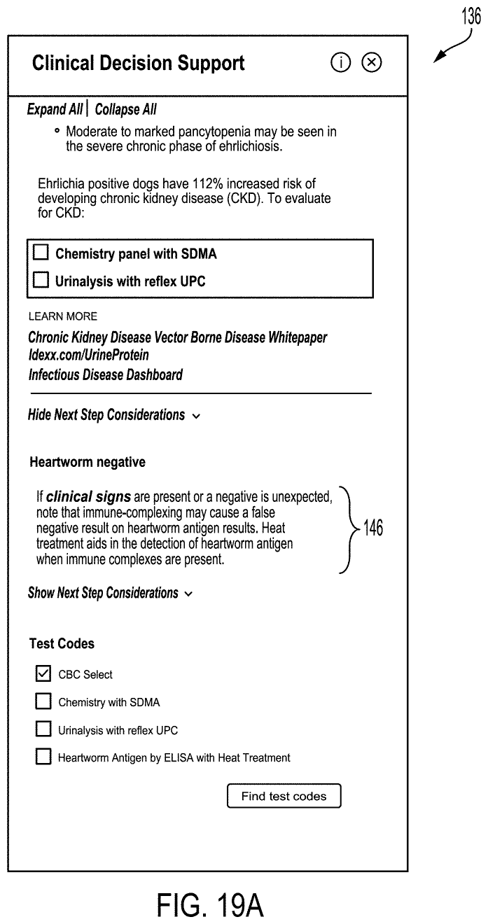

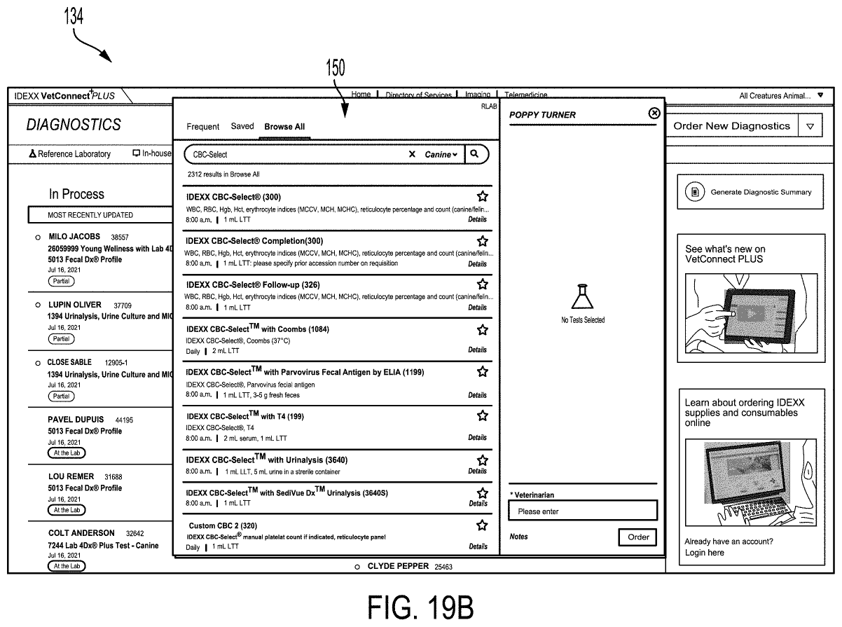

FIG. 19A illustrates another example of the clinical decision support interface 136 with details for test codes related to next step considerations, according to example implementations. For example, with reference to FIG. 18C, when a heartworm negative selection is input, next step considerations are presented for further possible diagnostic tests to conduct. In FIG. 19A, the clinical interpretation for the heartworm negative selection includes reference to a scenario in which if clinical signs are present or a negative is unexpected, an immune-complexing may cause a false negative result on the heartworm antigen results. Thus, further testing is recommended to assist with a diagnosis, which include tests such as CBC, chemistry panel, urinalysis, and a heartworm antigen with heat treatment. In FIG. 19A, a selection is input and received on the clinical decision support interface 136 (provided by the computing device 102) for the CBC test. Based on receiving the selection of the CBC test and selecting “Find test codes” in the clinical decision support interface 136, the computing device 102 accesses a database (such as the medical database 108 or patient information database 110 in FIG. 2) to retrieve information for input into an ordering module on the graphical user interface 134.

FIG. 19B illustrates an example of the graphical user interface 134 with an ordering module 150, according to an example implementation. The ordering module 150 is a graphical window that overlays information within the graphical user interface 134. The ordering module 150 is pre-populated with the patient information and a search bar is filled in with the test as selected in the clinical decision support interface 136, as shown in FIG. 19A. The ordering module 150 enables selection of a desired test or panel from a list generated due to the search for the selected test, and once the selection is received, an order is placed for the selected test. Thus, the clinical decision support interface 136 enables selection of a follow-on test, and the computing device 102 programmatically retrieves information of the patient and codes for use to identify the test from corresponding databases, and then triggers display of the ordering module 150 with a list of tests matching the selected codes.

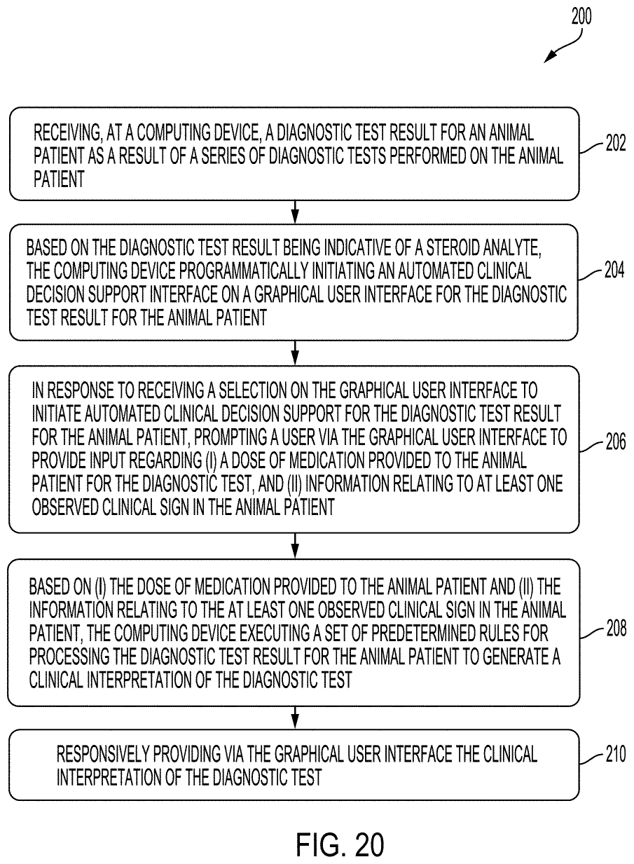

FIG. 20 shows a flowchart of an example of a method 200 for computer-implemented method for interpreting a diagnostic test result, according to an example implementation. Method 200 shown in FIG. 20 presents an example of a method that could be used with the system 100 shown in FIG. 1 or the computing device 102 shown in FIG. 2, for example. Further, devices or systems may be used or configured to perform logical functions presented in FIG. 10. In some instances, components of the devices and/or systems may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner. Method 200 may include one or more operations, functions, or actions as illustrated by one or more of blocks 202-210. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of present examples. In this regard, each block or portions of each block may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer- readable storage media in a machine-readable format, or on other non-transitory media or articles of manufacture. The computer readable medium may include non-transitory computer readable medium or memory, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In addition, each block or portions of each block in FIG. 20, and within other processes and methods disclosed herein, may represent circuitry that is wired to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

At block 202, the method 200 includes receiving, at the computing device 102, a diagnostic test result for an animal patient as a result of a series of diagnostic tests performed on the animal patient.

At block 204, the method 200 includes based on the diagnostic test result being indicative of a steroid analyte, the computing device programmatically initiating an automated clinical decision support interface 136 on the graphical user interface 134 for the diagnostic test result for the animal patient.

At block 206, the method 200 includes in response to receiving a selection on the graphical user interface 136 to initiate automated clinical decision support for the diagnostic test result for the animal patient, prompting a user via the graphical user interface 134 to provide input regarding (i) a dose of medication provided to the animal patient for the diagnostic test, and (ii) information relating to at least one observed clinical sign in the animal patient.

In some examples, input specific from the user can be avoided as such information may be included within the patient information database 112, and the computing device 102 may retrieve any required inputs from the patient information database 112, for example.

At block 208, the method 200 includes based on (i) the dose of medication provided to the animal patient and (ii) the information relating to the at least one observed clinical sign in the animal patient, the computing device 102 executing a set of predetermined rules 132 for processing the diagnostic test result for the animal patient to generate a clinical interpretation of the diagnostic test; and

At block 210, the method 200 includes responsively providing via the graphical user interface 134 the clinical interpretation of the diagnostic test.

In some further examples, the computing device 102 receives a notification from the patient information database 110 indicating that the animal patient received the treatment or additional testing, and then tracks compliance with the recommendation for treatment or additional testing for the animal patient.

In other examples, the computing device 102 monitors a stored profile of the animal patient (e.g., the patient profile 114) in the patient information database 110, and based on a change to the stored profile of the animal patient in the patient information database 110, tracks compliance with the recommendation for the treatment or additional testing for the animal patient.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to explain the principles of the examples, the practical application, and to enable others of ordinary skill in the art to understand the disclosure for various examples with various modifications as are suited to the particular use contemplated.

Different examples of the system(s), device(s), and method(s) disclosed herein include a variety of components, features, and functionalities. It should be understood that the various examples of the system(s), device(s), and method(s) disclosed herein may include any of the components, features, and functionalities of any of the other examples of the system(s), device(s), and method(s) disclosed herein in any combination or any sub-combination, and all of such possibilities are intended to be within the scope of the disclosure.

Thus, examples of the present disclosure relate to enumerated clauses (ECs) listed below in any combination or any sub-combination.

EC 1 is a computer-implemented method for interpreting a diagnostic test result, comprising receiving, at a computing device, a diagnostic test result for an animal patient as a result of a series of diagnostic tests performed on the animal patient, based at least in part on the diagnostic test result being indicative of a steroid analyte, the computing device programmatically initiating an automated clinical decision support interface on a graphical user interface for the diagnostic test result for the animal patient, in response to receiving a selection on the graphical user interface to initiate automated clinical decision support for the diagnostic test result for the animal patient, prompting a user via the graphical user interface to provide input regarding (i) a dose of medication provided to the animal patient for the diagnostic test, and (ii) information relating to at least one observed clinical sign in the animal patient, based at least in part on (i) the dose of medication provided to the animal patient and (ii) the information relating to the at least one observed clinical sign in the animal patient, the computing device executing a set of predetermined rules for processing the diagnostic test result for the animal patient to generate a clinical interpretation of the diagnostic test, and responsively providing via the graphical user interface the clinical interpretation of the diagnostic test.

EC 2 is the method of EC 1, further comprising providing for display, the graphical user interface, including a representation of the diagnostic test result for the series of diagnostic tests performed on the animal patient in rows and columns, and wherein the computing device programmatically initiating the automated clinical decision support interface on the graphical user interface for the diagnostic test result for the animal patient comprises, providing for display a side panel on the graphical user interface to prompt the user to provide the input, wherein the side panel overlays at least a portion of the representation of the diagnostic test result.

EC 3 is the method of any of ECs 1-2, wherein the computing device executing the set of predetermined rules for processing the diagnostic test result for the animal patient comprises receiving one or more characteristics of the animal patient selected from the group comprising: species, weight, and age, and generating the clinical interpretation of the diagnostic test based at least in part on the received one or more characteristics of the animal patient.

EC 4 is the method of any of ECs 1-3, further comprising filtering out possible clinical interpretations from clinical interpretations stored in memory based on the received one or more characteristics of the animal patient.

EC 5 is the method of any of ECs 1-4, wherein the diagnostic test result comprises a level of a hormone in the animal patient, and wherein the computing device executing the set of predetermined rules for processing the diagnostic test result for the animal patient comprises accessing, within a database, a set of clinical interpretations of the diagnostic test associated with an amount of the dose of medication provided to the animal patient, and mapping the diagnostic test result with one of the clinical interpretations in the set of clinical interpretations based at least in part on the level of the hormone in the animal patient being in a range of the level of hormone associated with the one of the clinical interpretations.

EC 6 is the method of any of ECs 1-5, further comprising determining whether the level of hormone in the animal patient is outside the range of the level of hormone associated with any of the clinical interpretations, in response to determining that the level of the hormone in the animal patient is outside of the range of the level of hormone associated with any of the clinical interpretations in the set of clinical interpretations: accessing patient test records for the animal patient within a patient information database, and generating the clinical interpretation of the diagnostic test by reference to the patient test records for the animal patient.

EC 7 is the method of any of ECs 1-6, further comprising generating a recommendation for treatment or additional testing based at least in part on the clinical interpretation, and responsively providing via the graphical user interface the recommendation.

EC 8 is the method of any of ECs 1-7, further comprising receiving a notification from a patient information database indicating that the animal patient received the treatment or the additional testing, and tracking, by the computing device, compliance with the recommendation for treatment or additional testing for the animal patient.

EC 9 is the method of any of ECs 1-8, further comprising monitoring, by the computing device, a stored profile of the animal patient in a patient information database, and based at least in part on a change to the stored profile of the animal patient in the patient information database, tracking, by the computing device, compliance with the recommendation for the treatment or additional testing for the animal patient.

EC 10 is the method of any of ECs 1-9, wherein receiving the diagnostic test result comprises receiving a test result of a dexamethasone suppression test.

EC 11 is the method of any of ECs 1-10, wherein prompting the user via the graphical user interface to provide input regarding the dose of medication provided to the animal patient for the diagnostic test comprises prompting the user via the graphical user interface to provide input regarding information indicating an amount of dexamethasone provided to the animal patient.

EC 12 is the method of any of ECs 1-11, wherein prompting the user via the graphical user interface to provide input regarding the information relating to at least one observed clinical sign in the animal patient comprises prompting the user via the graphical user interface to provide input regarding information indicating a presence or absence of a clinical sign consistent with Cushing's disease.

EC 13 is the method of any of ECs 1-12, further comprising displaying, via the graphical user interface, further possible diagnostic tests to conduct, receiving a selection on the graphical user interface for one of the further possible diagnostic tests, the computing device accessing a database to retrieve patient information for the animal patient and test code information for the one of the further possible diagnostic tests for input into an ordering module on the graphical user interface, and providing the ordering module as a graphical window that overlays information within the graphical user interface, wherein the ordering module is pre-populated with the patient information and includes a list of tests matching the test code information.

EC 14 is computing device comprising one or more processors, and non-transitory computer readable medium storing instructions executable by the one or more processors to perform functions comprising receiving a diagnostic test result for an animal patient as a result of a series of diagnostic tests performed on the animal patient, based at least in part on the diagnostic test result being indicative of a steroid analyte, initiating an automated clinical decision support interface on a graphical user interface for the diagnostic test result for the animal patient, in response to receiving a selection on the graphical user interface to initiate automated clinical decision support for the diagnostic test result for the animal patient, prompting a user via the graphical user interface to provide input regarding (i) a dose of medication provided to the animal patient for the diagnostic test, and (ii) information relating to at least one observed clinical sign in the animal patient, based at least in part on (i) the dose of medication provided to the animal patient and (ii) the information relating to the at least one observed clinical sign in the animal patient, executing a set of predetermined rules for processing the diagnostic test result for the animal patient to generate a clinical interpretation of the diagnostic test, and responsively providing via the graphical user interface the clinical interpretation of the diagnostic test.

EC 15 is the computing device of EC 14 wherein the diagnostic test result indicates a level of a hormone in the animal patient, and wherein executing the set of predetermined rules for processing the diagnostic test result for the animal patient comprises accessing, within a database, a set of clinical interpretations of the diagnostic test associated with an amount of the dose of medication provided to the animal patient, and mapping the diagnostic test result with one of the clinical interpretations in the set of clinical interpretations based on the level of the hormone in the animal patient being in a range of the level of hormone associated with the one of the clinical interpretations.

EC 16 is the computing device of any of ECs 14-15, wherein based on the level of the hormone in the animal patient being outside of the range of the level of hormone associated with any of the clinical interpretations in the set of clinical interpretations, the functions further comprise accessing patient test records for the animal patient within a patient information database, and generating the clinical interpretation of the diagnostic test by reference to the patient test records for the animal patient.

EC 17 is the computing device of any of ECs 14-16, wherein the functions further comprise generating a recommendation for treatment or additional testing based on the clinical interpretation, and responsively providing via the graphical user interface the recommendation.

EC 18 is the computing device of any of ECs 14-17, wherein the functions further comprise receiving a notification from a patient information database indicating that the animal patient received the treatment or additional testing, and tracking, by the computing device, compliance with the recommendation for treatment or additional testing for the animal patient.

EC 19 is a non-transitory computer readable medium having stored thereon instructions, that when executed by one or more processors of a computing device, cause the computing device to perform functions comprising receiving a diagnostic test result for an animal patient as a result of a series of diagnostic tests performed on the animal patient, based at least in part on the diagnostic test result being indicative of a steroid analyte, initiating an automated clinical decision support interface on a graphical user interface for the diagnostic test result for the animal patient, in response to receiving a selection on the graphical user interface to initiate automated clinical decision support for the diagnostic test result for the animal patient, prompting a user via the graphical user interface to provide input regarding (i) a dose of medication provided to the animal patient for the diagnostic test, and (ii) information relating to at least one observed clinical sign in the animal patient, based at least in part on (i) the dose of medication provided to the animal patient and (ii) the information relating to the at least one observed clinical sign in the animal patient, executing a set of predetermined rules for processing the diagnostic test result for the animal patient to generate a clinical interpretation of the diagnostic test, and responsively providing via the graphical user interface the clinical interpretation of the diagnostic test.

EC 20 is the non-transitory computer readable medium of EC 19, wherein the diagnostic test result indicates a level of a hormone in the animal patient, and wherein executing the set of predetermined rules for processing the diagnostic test result for the animal patient comprises accessing, within a database, a set of clinical interpretations of the diagnostic test associated with an amount of the dose of medication provided to the animal patient, and mapping the diagnostic test result with one of the clinical interpretations in the set of clinical interpretations based on the level of the hormone in the animal patient being in a range of the level of hormone associated with the one of the clinical interpretations.

EC 21 is the non-transitory computer readable medium of any of ECs 19-20, wherein based on the level of the hormone in the animal patient being outside of the range of the level of hormone associated with any of the clinical interpretations in the set of clinical interpretations, the functions further comprise accessing patient test records for the animal patient within a patient information database, and generating the clinical interpretation of the diagnostic test by reference to the patient test records for the animal patient.

EC 22 is the non-transitory computer readable medium of any of ECs 19-21, wherein the functions further comprise generating a recommendation for treatment or additional testing based on the clinical interpretation, and responsively providing via the graphical user interface the recommendation.

EC 23 is a computer-implemented method for interpreting diagnostic test results, comprising receiving, at a computing device, diagnostic test results for an animal patient as a result of a series of diagnostic tests performed on the animal patient, based on the diagnostic test results being indicative of an increased possibility of liver dysfunction, the computing device programmatically initiating an automated clinical decision support interface on a graphical user interface for the diagnostic test result for the animal patient, the computing device executing a set of predetermined rules to generate a hepatobiliary alert for inclusion in the automated clinical decision support interface, wherein executing the set of predetermined rules includes: dynamically generating complete blood count (CBC), urinalysis, and chemistry next step suggestions based on a lack of (CBC), urinalysis, and chemistry test results from tests performed on the animal patient within about a past month timeframe, dynamically generating a bile acids panel suggestion based on the diagnostic test results being indicative of the increased possibility of liver dysfunction, and creating the hepatobiliary alert including the complete blood count (CBC), urinalysis, and chemistry next step suggestions as well as the bile acids panel suggestion; and publishing the hepatobiliary alert in the automated clinical decision support interface.

EC 24 is the method of EC 23, wherein the bile acids panel suggestion includes information related to a testing protocol for use to conduct a bile acids panel diagnostic test on the animal patient.

EC 25 is the method of any of ECs 23-24, wherein based on receipt of bile acids panel diagnostic test results, responsively providing, via the graphical user interface, a clinical interpretation of the bile acids panel diagnostic test results.

By the term “substantially” and “about” used herein, it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide. The terms “substantially” and “about” represent the inherent degree of uncertainty that may be attributed to any quantitative comparison, value, measurement, or other representation. The terms “substantially” and “about” are also utilized herein to represent the degree by which a quantitative representation may vary from a stated reference without resulting in a change in the basic function of the subject matter at issue.

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure and should be interpreted in like manner as the more commonly used open-ended preamble term “comprising.”

What is claimed is:

1. A computer-implemented method for interpreting a diagnostic test result, comprising:
receiving, at a computing device, a diagnostic test result for an animal patient as a result of a series of diagnostic tests performed on the animal patient;
based at least in part on the diagnostic test result being indicative of a steroid analyte, the computing device programmatically initiating an automated clinical decision support interface on a graphical user interface for the diagnostic test result for the animal patient;
in response to receiving a selection on the graphical user interface to initiate automated clinical decision support for the diagnostic test result for the animal patient, prompting a user via the graphical user interface to provide input regarding (i) a dose of medication provided to the animal patient for the diagnostic test, and (ii) information relating to at least one observed clinical sign in the animal patient;
based at least in part on (i) the dose of medication provided to the animal patient and (ii) the information relating to the at least one observed clinical sign in the animal patient, the computing device executing a set of predetermined rules for processing the diagnostic test result for the animal patient to generate a clinical interpretation of the diagnostic test; and
responsively providing via the graphical user interface the clinical interpretation of the diagnostic test.

2. The computer-implemented method of claim 1, further comprising:
providing for display, the graphical user interface, including a representation of the diagnostic test result for the series of diagnostic tests performed on the animal patient in rows and columns; and
wherein the computing device programmatically initiating the automated clinical decision support interface on the graphical user interface for the diagnostic test result for the animal patient comprises, providing for display a side panel on the graphical user interface to prompt the user to provide the input, wherein the side panel overlays at least a portion of the representation of the diagnostic test result.

3. The computer-implemented method of claim 1, wherein the computing device executing the set of predetermined rules for processing the diagnostic test result for the animal patient comprises:
receiving one or more characteristics of the animal patient selected from the group comprising: species, weight, and age; and
generating the clinical interpretation of the diagnostic test based at least in part on the received one or more characteristics of the animal patient.

4. The computer-implemented method of claim 3, further comprising:
filtering out possible clinical interpretations from clinical interpretations stored in memory based on the received one or more characteristics of the animal patient.

5. The computer-implemented method of claim 1, wherein the diagnostic test result comprises a level of a hormone in the animal patient, and wherein the computing device executing the set of predetermined rules for processing the diagnostic test result for the animal patient comprises:
accessing, within a database, a set of clinical interpretations of the diagnostic test associated with an amount of the dose of medication provided to the animal patient; and
mapping the diagnostic test result with one of the clinical interpretations in the set of clinical interpretations based at least in part on the level of the hormone in the animal patient being in a range of the level of hormone associated with the one of the clinical interpretations.

6. The computer-implemented method of claim 5, further comprising:
determining whether the level of hormone in the animal patient is outside the range of the level of hormone associated with any of the clinical interpretations;
in response to determining that the level of the hormone in the animal patient is outside of the range of the level of hormone associated with any of the clinical interpretations in the set of clinical interpretations:
accessing patient test records for the animal patient within a patient information database; and
generating the clinical interpretation of the diagnostic test by reference to the patient test records for the animal patient. The computer-implemented method of claim 1, further comprising:
generating a recommendation for treatment or additional testing based at least in part on the clinical interpretation; and
responsively providing via the graphical user interface the recommendation.

8. The computer-implemented method of claim 7, further comprising:
receiving a notification from a patient information database indicating that the animal patient received the treatment or the additional testing; and
tracking, by the computing device, compliance with the recommendation for treatment or additional testing for the animal patient.

9. The computer-implemented method of claim 7, further comprising:
monitoring, by the computing device, a stored profile of the animal patient in a patient information database; and
based at least in part on a change to the stored profile of the animal patient in the patient information database, tracking, by the computing device, compliance with the recommendation for the treatment or additional testing for the animal patient.

10. The computer-implemented method of claim 1, wherein receiving the diagnostic test result comprises receiving a test result of a dexamethasone suppression test.

11. The computer-implemented method of claim 1, wherein prompting the user via the graphical user interface to provide input regarding the dose of medication provided to the animal patient for the diagnostic test comprises:
prompting the user via the graphical user interface to provide input regarding information indicating an amount of dexamethasone provided to the animal patient.

12. The computer-implemented method of claim 1, wherein prompting the user via the graphical user interface to provide input regarding the information relating to at least one observed clinical sign in the animal patient comprises:
prompting the user via the graphical user interface to provide input regarding information indicating a presence or absence of a clinical sign consistent with Cushing's disease.

13. The method of claim 1, further comprising:
displaying, via the graphical user interface, further possible diagnostic tests to conduct;
receiving a selection on the graphical user interface for one of the further possible diagnostic tests;
the computing device accessing a database to retrieve patient information for the animal patient and test code information for the one of the further possible diagnostic tests for input into an ordering module on the graphical user interface; and
providing the ordering module as a graphical window that overlays information within the graphical user interface, wherein the ordering module is pre-populated with the patient information and includes a list of tests matching the test code information.

14. A computing device comprising:
one or more processors; and
non-transitory computer readable medium storing instructions executable by the one or more processors to perform functions comprising:
receiving a diagnostic test result for an animal patient as a result of a series of diagnostic tests performed on the animal patient;
based at least in part on the diagnostic test result being indicative of a steroid analyte, initiating an automated clinical decision support interface on a graphical user interface for the diagnostic test result for the animal patient;
in response to receiving a selection on the graphical user interface to initiate automated clinical decision support for the diagnostic test result for the animal patient, prompting a user via the graphical user interface to provide input regarding (i) a dose of medication provided to the animal patient for the diagnostic test, and (ii) information relating to at least one observed clinical sign in the animal patient;
based at least in part on (i) the dose of medication provided to the animal patient and (ii) the information relating to the at least one observed clinical sign in the animal patient, executing a set of predetermined rules for processing the diagnostic test result for the animal patient to generate a clinical interpretation of the diagnostic test; and
responsively providing via the graphical user interface the clinical interpretation of the diagnostic test.

15. The computing device of claim 14, wherein the diagnostic test result indicates a level of a hormone in the animal patient, and wherein executing the set of predetermined rules for processing the diagnostic test result for the animal patient comprises:
accessing, within a database, a set of clinical interpretations of the diagnostic test associated with an amount of the dose of medication provided to the animal patient; and
mapping the diagnostic test result with one of the clinical interpretations in the set of clinical interpretations based on the level of the hormone in the animal patient being in a range of the level of hormone associated with the one of the clinical interpretations.

16. The computing device of claim 15, wherein based on the level of the hormone in the animal patient being outside of the range of the level of hormone associated with any of the clinical interpretations in the set of clinical interpretations, the functions further comprise:
accessing patient test records for the animal patient within a patient information database; and
generating the clinical interpretation of the diagnostic test by reference to the patient test records for the animal patient.

17. The computing device of claim 14, wherein the functions further comprise:
generating a recommendation for treatment or additional testing based on the clinical interpretation; and
responsively providing via the graphical user interface the recommendation.

18. The computing device of claim 14, wherein the functions further comprise:
receiving a notification from a patient information database indicating that the animal patient received the treatment or additional testing; and
tracking, by the computing device, compliance with the recommendation for treatment or additional testing for the animal patient.

19. A non-transitory computer readable medium having stored thereon instructions, that when executed by one or more processors of a computing device, cause the computing device to perform functions comprising:
receiving a diagnostic test result for an animal patient as a result of a series of diagnostic tests performed on the animal patient;
based at least in part on the diagnostic test result being indicative of a steroid analyte, initiating an automated clinical decision support interface on a graphical user interface for the diagnostic test result for the animal patient;
in response to receiving a selection on the graphical user interface to initiate automated clinical decision support for the diagnostic test result for the animal patient, prompting a user via the graphical user interface to provide input regarding (i) a dose of medication provided to the animal patient for the diagnostic test, and (ii) information relating to at least one observed clinical sign in the animal patient;
based at least in part on (i) the dose of medication provided to the animal patient and (ii) the information relating to the at least one observed clinical sign in the animal patient, executing a set of predetermined rules for processing the diagnostic test result for the animal patient to generate a clinical interpretation of the diagnostic test; and
responsively providing via the graphical user interface the clinical interpretation of the diagnostic test.

20. The non-transitory computer readable medium of claim 19, wherein the diagnostic test result indicates a level of a hormone in the animal patient, and wherein executing the set of predetermined rules for processing the diagnostic test result for the animal patient comprises:
accessing, within a database, a set of clinical interpretations of the diagnostic test associated with an amount of the dose of medication provided to the animal patient; and
mapping the diagnostic test result with one of the clinical interpretations in the set of clinical interpretations based on the level of the hormone in the animal patient being in a range of the level of hormone associated with the one of the clinical interpretations.

21. The non-transitory computer readable medium of claim 20, wherein based on the level of the hormone in the animal patient being outside of the range of the level of hormone associated with any of the clinical interpretations in the set of clinical interpretations, the functions further comprise:
accessing patient test records for the animal patient within a patient information database; and
generating the clinical interpretation of the diagnostic test by reference to the patient test records for the animal patient.

22. The non-transitory computer readable medium of claim 19, wherein the functions further comprise:
generating a recommendation for treatment or additional testing based on the clinical interpretation; and
responsively providing via the graphical user interface the recommendation.

23. A computer-implemented method for interpreting diagnostic test results, comprising:
receiving, at a computing device, diagnostic test results for an animal patient as a result of a series of diagnostic tests performed on the animal patient;
based on the diagnostic test results being indicative of an increased possibility of liver dysfunction, the computing device programmatically initiating an automated clinical decision support interface on a graphical user interface for the diagnostic test result for the animal patient;
the computing device executing a set of predetermined rules to generate a hepatobiliary alert for inclusion in the automated clinical decision support interface, wherein executing the set of predetermined rules includes:
dynamically generating complete blood count (CBC), urinalysis, and chemistry next step suggestions based on a lack of (CBC), urinalysis, and chemistry test results from tests performed on the animal patient within about a past month timeframe;
dynamically generating a bile acids panel suggestion based on the diagnostic test results being indicative of the increased possibility of liver dysfunction; and
creating the hepatobiliary alert including the complete blood count (CBC), urinalysis, and chemistry next step suggestions as well as the bile acids panel suggestion; and
publishing the hepatobiliary alert in the automated clinical decision support interface.

24. The computer-implemented method of claim 23, wherein the bile acids panel suggestion includes information related to a testing protocol for use to conduct a bile acids panel diagnostic test on the animal patient.

25. The computer-implemented method of claim 23, wherein based on receipt of bile acids panel diagnostic test results, responsively providing, via the graphical user interface, a clinical interpretation of the bile acids panel diagnostic test results.
